(19) United States
(12) Patent Application Publication
(10) Pub. No.: US 2023/0216086 A1
(43) Pub. Date: Jul. 6, 2023

(54) ELECTROLYTES FOR TARGET ION TRANSPORT

(71) Applicants: DEAKIN UNIVERSITY, Victoria (AU); MONASH UNIVERSITY, Victoria (AU)

(72) Inventors: Jenny PRINGLE, Victoria (AU); Karolina Matuszek, Victoria (AU); Tim Newbegin, Victoria (AU); Faezeh Makhlooghi Azad, Victoria (AU)

(21) Appl. No.: 17/924,827

(22) PCT Filed: May 13, 2021
(86) PCT No.: PCT/AU2021/050450

§ 371 Date: Nov. 11, 2022

(30) Foreign Application Priority Data
May 13, 2020 (AU) 2020901539

(51) Int. Cl. H01M 10/0564; H01M 10/052; H01M 10/054

(52) U.S. Cl. CPC H01M 10/0564; H01M 10/052; H01M 10/054; H01M 2300/0065

(57) ABSTRACT

The invention provides a zwitterionic plastic crystal (ZIPC) compound in the form of a single molecule comprising: at least one positively charged functional group carrying at least one positive charge, and at least one negatively functional group carrying at least one negative charge, wherein the positively charged functional groups and the negatively charged functional groups are covalently tethered together in the molecule, and the net charge of the zwitterionic compound is zero, and wherein the compound exhibits evidence of molecular disorder in the solid state.

### TECHNICAL FIELD

The invention relates to plastic crystal compounds having excellent target ion conduction ability and can be used in a variety of applications where fast target ion conduction is desired, for example, as electrolytes.

### BACKGROUND OF INVENTION

Plastic crystals are solids having a long-range, ordered crystal structure together with short-range disorder that originates from rotation or disorientation of individual molecules/ions within an ordered lattice. The short-range molecular rearrangements lead to the ability to deform under an applied load (i.e. plasticity) and to enhanced diffusivity of a second species within the plastic crystal lattice. Plastic crystal electrolytes can be classed as fast ion conductors, where a primary/target ion (e.g. Li⁺ for lithium batteries, or I⁻/I⁻₃ for dye-sensitised solar cells) moves rapidly against a background of a relatively static matrix.

The applicability of OIPCs as novel solid-state ion conductors in Li batteries, dye-sensitised solar cells, fuel cells, and Na batteries has been demonstrated recently. This is achieved by doping the OIPC with the appropriate cation, e.g. addition of Li salts for their application in Li batteries, or an acid or base for fuel cells. Furthermore, aprotic OIPCs offer good thermal and electrochemical stability and, due to their negligible volatility, significantly improve safety over present molecular solvent-based electrolytes. Organic Ionic Plastic Crystals (OIPCs) are structurally disordered salts that can exhibit soft, plastic mechanical properties and significant ionic conductivity. The structural disorder within OIPCs encourage fast target ion conduction when the OIPC is used as a matrix and a second component is introduced (e.g., an acid/base for a fuel cell, or Li or Na salts for Li/Na batteries) into the OIPC matrix and enables their use as solid electrolytes in electrochemical devices. However, their intrinsic structure (i.e. separate cations and anions) is considered to allow undesirable migration of the matrix OIPC ions. In an ideal electrolyte material, only the target ion (e.g., Li, Na, H) would migrate.

However, target ion transport through OIPCs is still not adequate and ultimately limits the achievable power output of a device. Indeed, the low transference number (fraction of the charge carried by the active species) e.g. t_{Li+} for OIPCs is commonly <0.2. This is due to the presence of other mobile species which carry charge, including the OIPC cation and anion, as well as the lithium salt counterion. For an ideal transference number (t_{LI+}=1), only Li ions should move through the electrolyte at any appreciable rate.

While zwitterionic liquids and even zwitterionic liquid crystals are known, in some rare cases, zwitterionic liquid crystals in combination with LiNTf₂ and propylene carbonate can be used as a liquid electrolyte, but leakage from the device, as well as the vapour pressure and flammability of this combination is problematic.

Organic ionic zwitterions in the electrochemistry arena have utilised sulfonate-based structures as these are relatively easy to synthesise in one step via a combination of a sulfone and methylpyrrolidine. However, these sulfonate zwitterions are crystalline solids that show no evidence of plasticity and thus are not suitable as a sole electrolyte matrix material as they do not have the soft mechanical properties required for battery cells. There is therefore an ongoing need for new electrolytes, which at least partially addresses one or more of the above-mentioned short-comings or provides a useful alternative.

Ohno et al (Phys. Chem. Chem. Phys., 2018, 20, 10978) describes an alkyl substituted imidazolium zwitterion ion that has a solid-solid transition at 165° C. below its Tₘ. However, there is no evidence that this zwitterion exhibits plastic behaviour as in addition to a lower entropy of melt, plastic zwitterions must exhibit evidence of disorder, preferably as determined by NMR studies. Furthermore, this compound is not used as a solid state electrolyte.

A reference herein to a patent document or any other matter identified as prior art, is not to be taken as an admission that the document or other matter was known or that the information it contains was part of the common general knowledge as at the priority date of any of the claims.

Where any or all of the terms “comprise”, “comprises”, “comprised” or “comprising” are used in this specification (including the claims) they are to be interpreted as specifying the presence of the stated features, integers, steps or components, but not precluding the presence of one or more other features, integers, steps or components.

### SUMMARY OF THE INVENTION

Prior to the present disclosure, it was not known that certain organic zwitterionic compounds exhibit plasticity as evidenced by molecular disorder in the solid state.

In a first aspect, the invention provides a zwitterionic plastic crystal (ZIPC) compound in the form of a non-polymeric molecule comprising:

at least one positively charged functional group carrying at least one positive charge, and

at least one negatively functional group carrying at least one negative charge, wherein

the positively charged functional groups and the negatively charged functional groups are covalently tethered together in the molecule, and the net charge of the zwitterionic compound is zero, and wherein the compound exhibits molecular disorder in the solid state, wherein the compound exhibits two or more of the following:

thermal phase behaviour which includes one or more solid-solid phase transitions before melting;
in the solid state one or more NMR linewidths of 20 KHz or less; and
a microstructure or morphology including slip and glide planes observable on SEM analysis. Desirably, the NMR linewidths are 10 KHz or less, preferably, 5 KHz or less, and in some embodiments are 1 KHz or less.

In a second aspect, the invention provides a zwitterionic plastic crystal (ZIPC) compound exhibiting molecular disorder in the solid state, having one of the general structures of claim 12.

In a third aspect, the invention provides a zwitterionic plastic crystal (ZIPC) compound exhibiting molecular disorder in the solid state, having one of the structures of claim 13.

In a fourth aspect, the invention provides a compound exhibiting molecular disorder in the solid state, having one of the following structures:

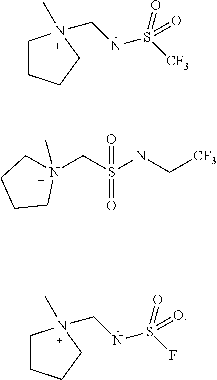

In a fifth aspect, the invention provides a use of a compound of the first to fourth aspects as a solid-state solvent.

In a sixth aspect, the invention provides a use of a compound the first to fourth aspects as an electrolyte matrix, preferably a solid state electrolyte matrix.

In a seventh aspect, the invention provides a use of a compound according to the first to fourth aspects, in an electrolyte as a conductivity enhancing additive, preferably wherein the electrolyte is a polymer based electrolyte or an ionic liquid based electrolyte.

In an eight aspect, the invention provides a method of identifying a zwitterionic plastic crystal (ZIPC) compound comprising the steps of:

(i) providing a non-polymeric zwitterionic compound comprising: at least one positively charged functional group carrying at least one positive charge, and at least one negatively functional group carrying at least one negative charge, wherein the positively charged functional groups and the negatively charged functional groups are covalently tethered together in the molecule, and the net charge of the zwitterionic compound is zero,

(ii) establishing the zwitterionic compound as a zwitterionic plastic crystal (ZIPC) compound by screening the zwitterionic compound for evidence of molecular disorder in the solid state which identifies the zwitterionic compound as a zwitterionic plastic crystal (ZIPC) compound, wherein molecular disorder is evidenced by the compound exhibiting two or more of the following:

thermal phase behaviour which includes one or more solid-solid phase transitions before melting;
in the static solid state NMR spectra, one or more NMR linewidths of 20 KHz or less; and
a microstructure or a morphology including slip and glide planes on SEM analysis. Desirably, the NMR linewidths are 10 KHz or less, preferably, 5 KHz or less, and in some embodiments are 1 KHz or less.

In a ninth aspect, the invention provides a zwitterionic plastic crystal (ZIPC) compound obtainable by the method of the eighth aspect.

In a tenth aspect, the invention provides a zwitterionic plastic crystal composition in liquid form comprising a zwitterionic plastic crystal (ZIPC) compound according to the first to fourth aspects or the ninth aspect, and an ionic salt, an acid, a base, a Li or Na functionalised polymer or combinations thereof.

In an eleventh aspect, the invention provides a zwitterionic plastic crystal composition in a solid-state form comprising a zwitterionic plastic crystal (ZIPC) compound according to the first to fourth aspects or the ninth aspect, and an ionic salt, an acid, a base or a Li or Na functionalised polymer or combinations thereof.

In a twelfth aspect, the invention provides a use of a zwitterionic plastic crystal (ZIPC) compound according to the first to fourth aspects or the ninth aspect, or a zwitterionic plastic crystal (ZIPC) composition according the ninth or tenth aspects, in an application requiring ion conduction, for example, an electrochemical cell, including an electrochemical device, preferably a fuel cell, a supercapacitor, dye-sensitised solar cell or an energy storage device such as a Na battery or a Li battery.

In a thirteenth aspect, the invention provides a use of a zwitterionic plastic crystal (ZIPC) compound according to the first to fourth aspects or the ninth aspect in a protic form in an application requiring proton conduction, for example, a fuel cell.

In a fourteenth aspect, the invention provides a use of a base doped zwitterionic plastic crystal (ZIPC) compound according to the first to fourth aspects or the ninth aspect as an anhydrous proton conductor, preferably wherein the base is imidazole.

In a fifteenth aspect, the invention provides a solid-state electrolyte comprising a zwitterionic plastic crystal (ZIPC) compound according to the first to fourth aspects or the ninth aspect.

In a sixteenth aspect, the invention provides a solid-state electrolyte comprising the solid-state composition of the tenth or eleventh aspects.

In a seventeenth aspect, the invention provides a use an energy storage device, comprising an electrolyte comprising a zwitterionic plastic crystal (ZIPC) matrix, optionally doped with an ionic salt, an acid, a base, a Li or Na functionalised polymer or combinations thereof.

In an eighteenth aspect, the invention provides a use an energy storage device according to the seventeenth aspect, wherein the energy storage device is a Na battery or a Li battery.

In a nineteenth aspect, the invention provides a fuel cell device comprising an electrolyte comprising a zwitterionic plastic crystal (ZIPC) matrix, optionally doped with an ionic salt, an acid, a base, a Li or Na functionalised polymer or combinations thereof.

### BRIEF DESCRIPTION OF DRAWINGS

Embodiments of the invention will herein be illustrated by way of example only with reference to the accompanying drawings in which:

FIG. 1-1A illustrates the structures of a number of new zwitterionic plastic crystals (ZIPCs), with comparison to a number of analogous established OIPCs. Compounds 1, 2, 5 and 6 are novel compounds made on request via a custom synthesis by Boron Molecular. Novel compounds 3 and 4 are made at Deakin University. Compounds 7,8 and 9 are commercially available but have not previously been described as plastic crystals.; and 1B illustrates thermal analysis information for pure ZIPC1, ZIPC2, ZIPC5 and ZIPC6. FIG. 1C shows cations and anions for combination to form ZIPCs

FIGS. 2-2A illustrates the differential scanning calorimetry (DSC) heating traces of (a) ZIPC1 and 10 mol% LiFSI doped ZIPC1; and (b) Pure [C₂mpyr][BF₄] OIPC and 10 mol % LiFSI doped [C₂mpyr][BF₄] OIPC. The heating/cooling rate is ±10 K/min; 2B illustrates DSC heating traces of (a) ZIPC1 and an electrolyte mixture of 90 mol % LiFSI in ZIPC1;

FIGS. 3-3A illustrates SEM images and microstructures of (a) pure ZIPC1 (as a pellet); and (b) 10 mol % Li FSI in ZIPC1; 3B illustrates SEM images of an electrolyte mixture of 90 mol % LiFSI in ZIPC1; 3C illustrates a SEM image of ZIPC6. (Pellets were prepared and images captured at room temperature); 3D illustrates SEM image of the surface of NaF; 3E illustrates a SEM image of pure ZIPC5 pressed into a pellet at room temperature;

FIGS. 4-4A illustrates the ionic conductivity of pure ZIPC1 and [C₂mpyr][BF₄] OIPC and their mixtures with LiFSI as a function of temperature; FIG. 4Aa is ionic conductivity of 10 mol % LiFSI in ZIPC1 and OIPC. FIG. 4Ab is linewidth of them vs. temperature. FIG. 4Ac ionic conductivity of pure ZIPC, OIPC and their mixture with 10 mol % LiFSI; 4B illustrates ionic conductivity of pure ZIPC1 and an electrolyte mixture of 90 mol % LiFSI in ZIPC1 as a function of temperature;

FIGS. 5-5A illustrates variable temperature-static ⁷Li spectra of (a) 10 mol % LiFSI doped OIPC; (b) 10 mol % LiFSI doped ZIPC1; and (c) comparison of ⁷Li Linewidth as a function of temperature; 5B illustrates (a) Variable temperature-static ⁷Li spectra of a 90 mol % LiFSI and ZIPC1 electrolyte mixture; (b) VT-static ⁷Li spectra of pure LiFSI; (c) VT-static ¹⁹F spectra of 90 mol % LiFSI and ZIPC1 electrolyte mixture; and (d) ⁷Li and ¹⁹F linewidth of 90 mol % LiFSI and ZIPC1 electrolyte mixture as a function of temperature; 5C(a) illustrates ¹H single-pulse spectra of pure ZIPC5; 5C(b) illustrates ¹⁹F single-pulse spectra, of pure ZIPC 5 versus temperature; 5D(a) Ionic conductivity of 10 mol % of LiFSI in ZIPC5; 5D(b) The DSC trace of 10 mol % LiFSI in ZIPC5; 5D(c) The SEM images 10 of LiFSI in ZIPC5;

FIG. 6 illustrates a) VT-static ¹⁹F spectra of 10 mol % LiFSI doped ZIPC1 and 10 mol % LiFSI doped OIPC at 20° C. and 60° C.; and b) ¹⁹F line width of BF₄ in OIPC and BF₃ in ZIPC1; c) ¹⁹F linewidth of FSI, as a function of temperature; d) The single pulsed 19F spectra for ZIPC1 as a function of temperature are shown. Note that for a crystalline solid, the linewidths would be very broad >100 ppm; e) illustrates (i) NMR line widths of ZIPC1 as a function of temperature for 19F; (ii) Area fractions of narrow peaks for 19F in ZIPC1 as function of temperature, obtained from deconvolution of the ¹⁹F static NMR spectra. The blue dashed lines separate different thermal phases, as determined by DSC.

FIGS. 7-7A illustrates a comparison of ⁷Li, ¹⁹F and ¹H diffusion coefficients of 10 mol % LiFSI doped ZIPC1 and 10 mol % LiFSI doped OIPC at different temperatures measured by PFG-NMR. Red plots are OIPC and black ones are ZIPC1; 7B illustrate ⁷Li (black) and ¹⁹F (red) diffusion coefficients of a 90 mol % LiFSI and ZIPC1 electrolyte mixture at different temperatures measured by PFG-NMR;

FIG. 8 illustrates cyclic voltammogram of 10 mol % LiFSI doped ZIPC1 at 0.05 mV S⁻¹ at 50° C.;

FIGS. 9-9A illustates chronoamperometry of Li|10 mol % LiFSI doped ZIPC1 electrolyte|Li cells at a potential step of 10 mV at 50° C.; 9B illustrates chronoamperometry of Li|90 mol % LiFSI in ZIPC1 electrolyte mixture|Li cells at a potential step of 10 mV at 50° C.;

FIGS. 10-10A illustrates a) Li|Li symmetric cell cycling of 10 mol % LiFSI in ZIPC1 at different current densities with 1 hour polarisation time (10 cycles at each current density) at 50° C.; —10B illustrates symmetric cell cycling performance of a 10 mol % LiFSI and ZIPC1 electrolyte mixture at 0.1 mA/cm² at 50° C.;

FIG. 11 illustrates cycling performance of (lithium iron phosphate) LFP|10 mol % LiFSI in ZIPC|1 Li at 50° C. in the range of 2.8 to 3.8 V;

FIG. 12 illustrates that a DSC trace of ZIPC7 shows 3 peaks in the heating cycle (T1=92° C.; ΔH=26 J/g; T2=106° C.; ΔH=10 J/g; T3=119° C.; ΔHf=25 J/g) (Melting point of imidazole =89 ° C.) and the effect of imidazole doping at different concentrations;

FIG. 13 illustrates a) conductivity of pure protic ZIPC7, and upon doping with different amounts of imidazole base. The conductivity of each sample was measured in triplicate.

FIG. 14 illustrates results for the conductivity of the triflic acid doped protic ZIPC7.

FIG. 15 illustrates conductivity and symmetrical lithium cell performance of the liquid 50 mol % LiFSI in ZIPC1 electrolyte; (a) Ionic conductivity and viscosity (inset—DSC trace of the liquid electrolyte) b) Li|Li symmetrical cell voltage profile at 50° C. at 0.2 mA cm⁻² polarisation time 1 h per step c) Li|Li symmetrical cell cycling at different current densities at 50° C., polarisation time 1 h per step; and

FIG. 16 illustrates DSC traces of pure ZIPC1 and its mixtures with 10 and 90 mol % LiBF₄, b and c) SEM images of 10 mol % and 90 mol % LiBF₄ in ZIPC1 respectively, d and e) ⁷Li single-pulse static NMR spectra versus temperature of 10 mol % and 90 mol % LiBF₄ in ZIPC1, respectively. f and g) ¹⁹F single-pulse static NMR spectra versus temperature of 10 mol % and 90 mol % LiBF₄ in ZIPC1, respectively;

FIG. 17 illustrates a) Ionic conductivity of pure ZIPC1 and its mixtures with 10 and 90 mol % LiBF₄, b) ⁷Li and ¹⁹F diffusion coefficients for 10 and 90 mol % LiBF₄ in ZIPC1 at different temperatures; and

FIG. 18 illustrates cyclic voltammograms of a) 10 and b) 90 mol % LiBF₄ in ZIPC1 at 50° C., collected at a scan rate of 0.05 mV s⁻¹ using a stainless-steel working electrode versus a Li metal reference electrode.

### DETAILED DESCRIPTION OF THE INVENTION

The inventors have unexpectedly discovered that covalently tethering together certain cations and anions, preferably from an OIPC, can form a zwitterionic plastic crystal (ZIPCs) compound. Such tethering of ions reduces/eliminates the net matrix ion migration observed for OIPCs in an electric field. Ion tethering increasing transport of a target ion through the ZIPC, e.g., through a ZIPC electrolyte matrix doped with a source of target ion. Plastic zwitterions address low target ion transference number problems observed in existing solid state electrolyte matrices (e.g., OIPC electrolyte matrix, which results from translational migration of the OIPC matrix ions). The solution involves eliminating undesirable migration of the matrix OIPC ions by using a ZIPC matrix in which the positive and negative charges are tethered together in a net neutral molecule which does not move in an electrical field while the unexpected ZIPC plasticity (arising from the surprising retention of overall disorder in the ZIPC) enables high target ion conductivity, when the ZIPC electrolyte matrix is doped with a salt of the target ion. It was not expected that tethering charges in a single molecule would have provided these benefits as tethering would have been expected to reduce the opportunities for rotation and translation disorder. It was unexpected that certain ion tethered compounds exhibit plasticity and that the ZIPCs of the invention would exhibit sufficient disorder to enable better target ion transport in a solid state matrix. No previous studies suggest that tethering ions of an organic ionic plastic crystal would to form a plastic zwitterion as such ion tethering would have been expected to produce a typically ordered crystalline compound since ion tethering would be expected to reduce the number of possible disordering motions (rotational and translational), thereby teaching away from the zwitterionic plastic crystal compounds of the invention. Furthermore, such a matrix would be expected to lack usefulness in terms of assisting dissociation of a target ion from a salt provided to the electrolyte matrix. Nothing in the art suggests that the zwitterions described would demonstrate better target ion transference numbers than a corresponding OIPC.

The ZIPCs of the invention offer improved solid-state conductivity and transport of target ions (e.g., Li+, Na+, H+), while simultaneously suppressing counter ion transport, which has been a significant challenge with OIPCs. This is demonstrated by the high transference number, e.g., (t_{Li+}) of 0.7 for a 90 mol % LiFSI in ZIPC1 solid electrolyte mixture. Typical transference numbers for Li salts in OIPCs are <0.2. The ZIPCs are particularly suited to use in cells with metal anodes, for example, lithium or sodium metal anodes.

Furthermore, the protic and aprotic ZIPCs provide improvements in proton conductivity over protic and aprotic OIPCs.

ZIPCs are a new class of materials proposed as (i) a solid state electrolyte matrix material to be doped with salts, particularly Li⁺ or Na⁺ containing materials for batteries, (ii) as additives for other electrolytes to facilitate dissociation and transport of target ions, particularly Li⁺ or Na⁺ ions, (iii) as proton conducting materials for proton exchange membrane (PEM) fuel cells, when doped with acid or base; and/or (iv) as a replacement for OIPCs in existing ion conductor applications. In terms of (i), the new ZIPC electrolyte materials have high ionic conductivities of >10⁻⁹ S cm⁻¹ and t_{Li+}>0.2 when doped with lithium salt.

In Li and Na batteries, the ZIPCs of the invention can be used as additives within other electrolytes, such as polymer based electrolytes or ionic liquid based electrolytes to encourage target ion dissociation and enhanced mobility of the target ion and transport through the electrolyte. This can be achieved by the ZIPC providing another (charge diffuse) negatively charged site to interact with the positively charged target ion (e.g. Li⁺ or Na⁺), competing with the interactions between the Li⁺ and its counterion from the salt, thereby increasing ion dissociation.

In the field of polymer-based solid-state electrolytes, the ZIPCs as additives improve dissociation of the charge carrier ions from the polymer backbone (or other ionic species present).

Using the ZIPCs in analogous OIPC applications may advantageously result in higher conductivities of specific target ions.

Furthermore, the ZIPCs of the invention, particularly those having zwitterionic-BF₃ structures, may be less prone to hydrolysis than the equivalent BF₄ species. As such, even though in battery applications, the electrolytes are generally used under an inert atmosphere, using the ZIPC compounds in a device comprising an electrochemical cell may advantageously provide for longer term device/cell stability due to the possibility of less of a tendency toward hydrolysis. This may be particularly important for fuel cells.

The inventors have extended the concept to protonated zwitterions (with a mobile proton) and have demonstrated the protonated ZIPCs enable good proton conduction.

Suitably, preferred ZIPCs are non-volatile. Desirably, preferred compounds are not flammable or explosive, at least under the typical operating conditions of a fuel cell or energy storage device.

Suitably, the ZIPC compounds exhibit a long range, ordered crystal structure together with short-range disorder that originates from rotation or disorientation of the molecules within an ordered lattice. For ZIPCs, solid-solid phase transitions are understood to be associated with the onset of rotational motion of all or parts of the ZIPC molecule. A combination of spectroscopic and modelling approaches can be a powerful way to further elucidate the interplay between chemistry, structure, and phase behaviour in ZIPCs and can serve as predictors of plastic behaviour in zwitterions as described herein.

Molecular disorder associated with a ZIPC (and thus plasticity) can observed for example from characteristic features in at least two or more of thermal studies, solid-state NMR studies and SEM studies. Notably, one or more of the characteristics features can increase with increasing temperature.

One characterising feature can include thermal phase behaviour which includes one or more solid-solid phase transitions before melting (a pre-melting or sub-melting solid-solid phase transition). Techniques for measuring and characterising a solid-solid phase transition of a ZIPC include Differential Scanning Calorimetry whereby a solid-solid phase transition is characterised by a DSC plot in which a discontinuity (e.g. a spike) of the heat flow in the sub-melting temperature range is observed which is in addition to, and distinct from, the discontinuity arising from the solid-liquid (melting) transition of the ZIPC.

Another characterising feature of molecular disorder in the solid state is determined from static solid-state NMR, whereby plastic ZIPCs exhibit one or more NMR linewidths of 20 KHz or less. Desirably, the linewidths narrow further with increasing temperature. Desirably, the NMR linewidths are 10 KHz or less, preferably, 5 KHz or less, and in some embodiments are 1 KHz or less.

Another characterising feature of molecular disorder in the solid state is determined by the observations on the microstructure/morphology by SEM analysis. Characteristic features include observations of several grains with different orientations, observation of slip and glide planes on SEM analysis, sets of slip planes within different grains, observation of grain boundaries from fractured surfaces of the material. Further evidence of plasticity increases with increasing temperature.

Another characterising feature can include exhibition of an entropy of fusion, ΔS_{f} of less than about 60 JK⁻¹ ⁻¹, more preferably less than about 50 JK⁻¹ mol⁻¹, more preferably less than about 40 JK⁻¹ mol⁻¹, more preferably less than about 30 JK⁻¹ mol⁻¹, more preferably less than about 20 JK⁻¹ mol⁻¹.

Other useful studies include X-ray diffraction, Raman spectroscopy, synchrotron X-ray diffraction and molecular modelling such a molecular dynamics (MD) or combinations thereof.

Preferred ZIPC compounds are plastic solids at application operation temperatures, for example at about −100° C. to about 200° C., at about −50° C. to about 100° C., most preferably at about −10° C. to about 80° C. Particularly preferred compounds are plastic solids at least at room temperature. By ‘room temperature’ it is meant a temperature of from about 20° C. to about 25° C., preferably 25° C. Preferred ZIPC compounds have a melting point≥60° C., ≥70° C., ≥80° C., ≥80° C., ≥100° C., ≥150° C., ≥200° C. or ≥250° C. Preferred compounds exhibit plastic behaviour at temperatures of from about −100° C. to about 100° C. The melting point dictates the upper normal operating temperature of a device using a ZIPC. By ‘melting point’, it is meant the extrapolated onset temperature associated with a phase transition on melt from a solid to a liquid as determined by differential scanning calorimetry (DCS). Where a compound exhibits plasticity at very low temperatures, e.g., <0° C., typically it indicates that the compound will be advantageously very disordered at room temperature.

It is thought that when used as electrolytes, plastic crystals provide an environment through which added target ions can move, e.g., through vacancies, grain boundaries and/or the formation of additional liquid, liquid-like amorphous phases. Indeed, SEM analysis of a number of electrolyte materials comprising lithium salts show crystalline regions and intergranular regions contain mobile, Li rich electrolyte providing pathway for lithium ions which supports the lithium electrochemistry and device cycling. Thus, it is believed that the solid materials of the invention composed of ZIPC and doped salt comprise one or more of a liquid or a liquid-like phase or an amorphous phase, for example, that is rich in salt. Thus, the materials comprise more than one phase. It is thought that a target ion rich liquid or a target ion rich liquid-like phase or a target ion rich amorphous phase provides pathways for target ion diffusion and facilitates target ion transport through the electrolyte.

In some cases where the functional group carrying the positive charge comprises an alkyl chain, one or more of the melting and the Phase II-I transition temperature may decrease with increasing alkyl chain length.

ZIPCs can be categorised into protic and aprotic classes depending on the availability of dissociable proton on the cationic and/or the anionic component of the zwitterionic molecule. Thus, some suitable cations may be protic or aprotic cations, depending on the availability of labile proton(s). Likewise, some suitable anions may be protic or aprotic anions, depending on the availability of labile proton(s).

ZIPC formation—ZIPCs can be provided starting from at least one cation and at least one anion and covalently tethering these together. Provided the combination of cations and anions tethered together provide (i) a zwitterionic compound with a net neutral electrical charge, (ii) is a plastic crystal exhibiting molecular disorder (and thus plasticity), for example, which can observed from characteristic features in two or more of thermal studies, solid-state NMR studies and SEM studies, there is no particular limitation on the type of cations and associated counter anions that can be employed.

In preferred ZIPC compounds, at least one of the positive functional groups of the ZIPC is derived from a small cationic component, such as an optionally substituted saturated or unsaturated heterocyclic ring, for example, pyrrolidine, morpholinium, piperidinium, thiolane, benzotriazole or tetrahydrofuran. Desirably, at least one of the negative functional groups of a preferred ZIPC is derived from a charge delocalizing anionic group such as fluoroborate, oxalatoborate, sulfonylimide, fluorosulfonylimide (FSI), bis(trifluoromethanesulfonyl)imide (TFSA). By ‘derived from’, it is meant that the respective cation or anion form the basis of a corresponding functional groups which are covalently bonded together, directly or through at least one atom or intermediate functional group which can be, for example, a carbon bond or hydrocarbon chain or indeed an additional functional group, ring or chain. It will be understood that, as a result of the tethering of the functional groups together in the ZIPC molecule, the corresponding functional groups derived from the cations and anions are not readily dissociable from each other, particularly under the influence of an electric field.

Cation component for tethering—Some suitable cations may be di-cations or tri-cations. Preferred cations are symmetrical. In some embodiments, the cation is a chiral cation.

Examples of suitable cations include pyrrolidinium, imidazolium, phosphonium, metallocenium cations, which can be unsubstituted or substituted with one or more functional groups selected from C₁₋₆ alkyl, preferably methyl, ethyl or propyl, CN, OMe, OEt and CN.

Suitably, one or more of the functional groups carrying a positive charge can be selected from the group of cations and particularly aprotic cations, consisting of: Cₙ(N_{2,2,m})₂ where n=2, 3, 4, 6 and m=1, 2, 3, 4, 6; N_{2,1,1,1}; N_{2,2,1,1}; N_{2,2,2,1}; N_{2,3,3,3}; N_{2,2,3,3}; N_{2,2,2,3}; N_{4,4,4,4}; P_{1,2,2,2}; N_{1,2,3,i3}; N_{2,2,2,2}; N_{3,3,3,3} and C₂epyr. Cations that are capable of rotational motions (e.g., tetramethylammonium) are particularly desirable.

Desirably, at least one of the positively charged functional group carrying at least one positive charge is derived from an ammonium cation, a phosphonium cation or a sulfonium cation, which contain a nitrogen having a positive charge, a phosphorus having a positive charge, and a sulfur having a positive charge respectively.

Desirably, at least one positively charged functional group carrying at least one positive charge is derived from an ammonium cation which contains nitrogen and has a positive charge. A preferred ammonium cation may have general formula [NR⁴R³R²R¹]⁺. Desirably, at least one positively charged functional group carrying at least one positive charge is derived from a sulfonium cation which contains sulfur and has a positive charge. A preferred sulfonium cation may have general formula [SR³R²R¹]⁺. Desirably, at least one positively charged functional group carrying at least one positive charge is derived from a phosphonium cation which contains phosphorus and has a positive charge. A preferred phosphonium cation may have general formula [PR⁴R³R²R¹]⁺.

In each case above, each of R¹ to R⁴ may be the same or different and may be independently selected from optionally substituted alkyl and optionally substituted aryl, or where one R group is selected from optionally substituted alkyl and optionally substituted aryl and the remaining two R groups together with P form an optionally substituted heterocyclic ring, and R¹ is selected from H, optionally substituted alkyl, and optionally substituted aryl. Examples of suitable phosphonium cations include tetra(C₁₋₂₀alkyl) phosphonium, tri(C₁₋₉alkyl) mono(C₁₀₋₂₀alkyl) phosphonium, tetra(C₆₋₂₄aryl) phosphonium, phospholanium, phosphinanium and phosphorinanium.

Desirably, at least one of the positively charged functional groups carrying at least one positive charge is derived from a morpholinium cation, a pyrrolidinium cation or an imidazolium, each of which contain nitrogen having a positive charge. The ring of the pyrrolidinium cation or an imidazolium may be unsubstituted or substituted with one or more of R¹ and R². In each case, each of R¹ and R² may be the same or different and may be independently selected from optionally substituted alkyl and optionally substituted aryl, or where one R group is selected from optionally substituted alkyl and optionally substituted aryl and the remaining two R groups together with P form an optionally substituted heterocyclic ring, and R¹ is selected from H, optionally substituted alkyl, and optionally substituted aryl.

Other preferred cations for tethering to suitable anions include dialkylpyrrolidinium, pyrrolidinium, monoalkylpyrrolidinium, dialkylimidazolium, monoalkylammonium, imidazolium, tetraalkylammonium, quaternary ammonium, trialkylammonium, dialkylammonium, dialkylammonium, dialkanolalkylammonium, alkanoldialkylammonium, bis(alkylimidazolium), bis(dialkyl)ammonium, bis(trialkyl)ammonium, diallylammonium, dialkanolammonium, alkylalkanolammonium, alkylallylammonium, guanidinium, diazabicyclooctane, tetraalkyl phosphoniums, trialkylphosphoniums, trialkylsulfoniums, tertiarysulfoliniums, imidazolinium, cholinium, formamidinium, formadinium, bicyclic (spiro) ammonium, pyrazolium, benzimidazolium, dibenzylammonium, caffineium, piperazinium, diallyl(amino)ammonium, alkyl(diamino)ammonium, triaminoammonium, aminopyrrolidium, and aminoimidazolium.

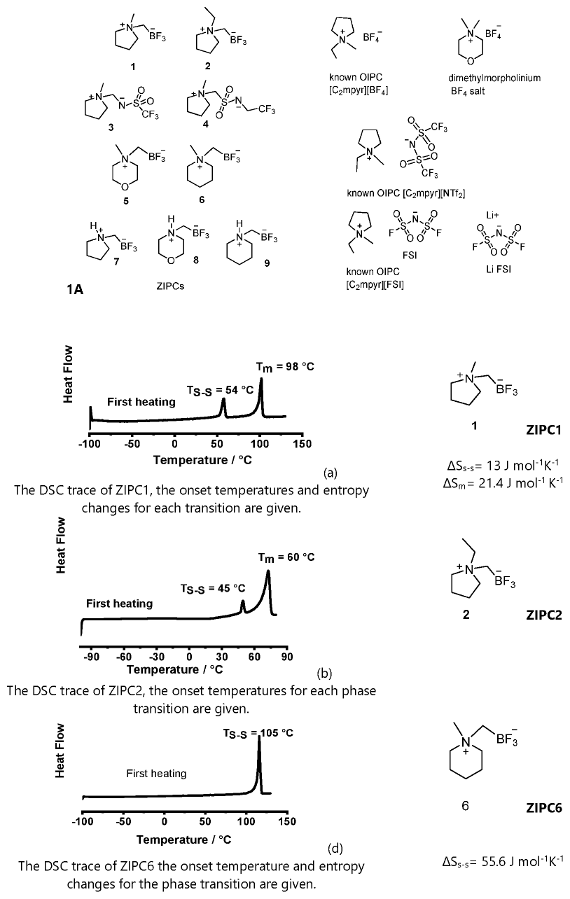

Other cations for tethering may be selected from the group consisting of the cations illustrated in FIG. 1C.

In one embodiment, desirably, at least one of the positively charged functional groups carrying at least one positive charge is derived from a cation from an ionic liquid or more preferably from an OIPC. Desirably, at least one of the negative charged functional groups carrying at least one negative charge is derived from an anion from an ionic liquid or an OIPC. Suitably, the ZIPC of the invention may be formed by tethering together in the same molecule at least one cation from an ionic liquid or an OIPC and the at least one anion from an ionic liquid or an OIPC. The skilled synthetic chemist will be able to devise suitable synthetic methodologies to form compounds in which the desired groups are tethered together.

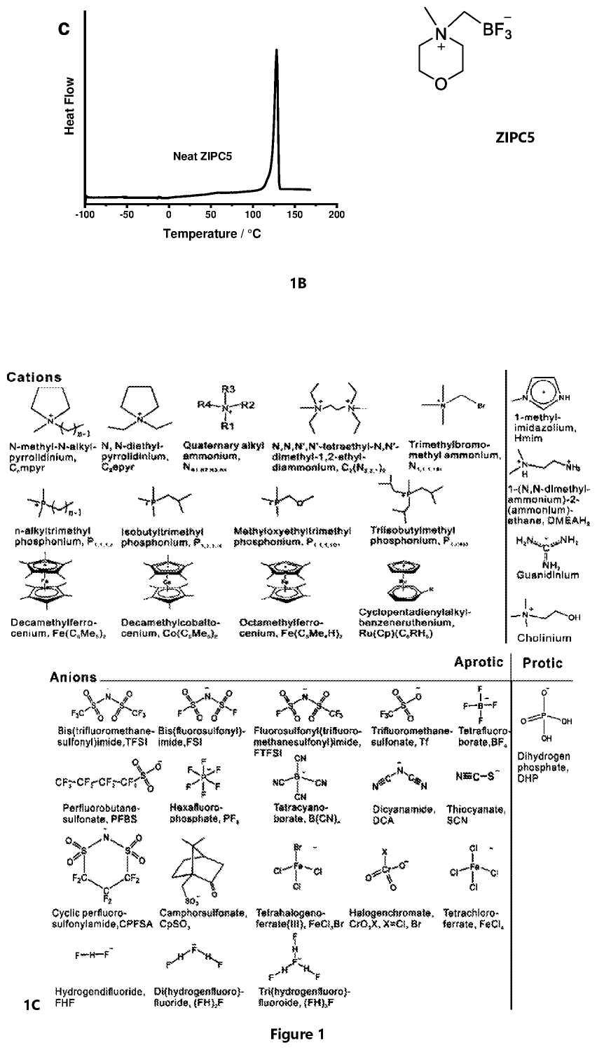

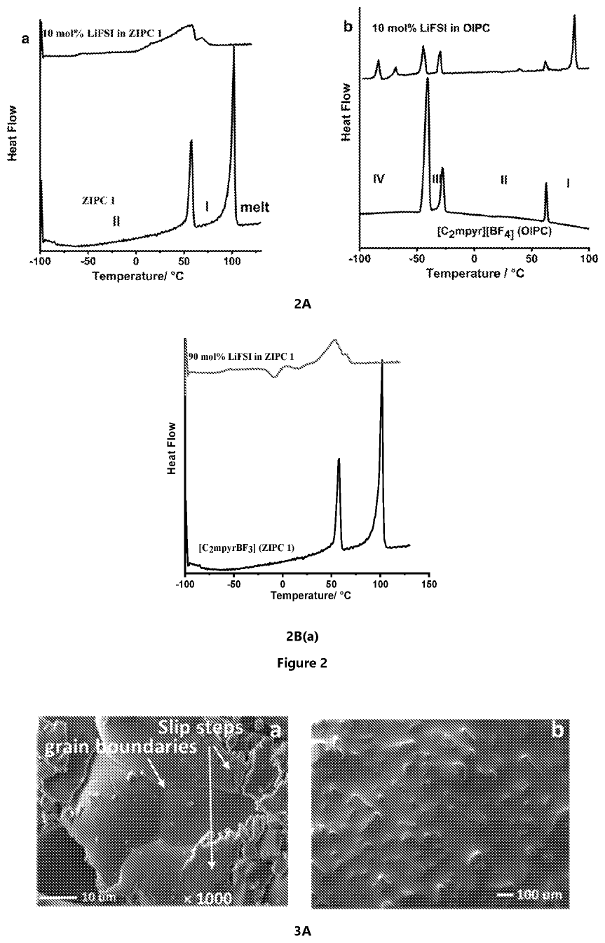

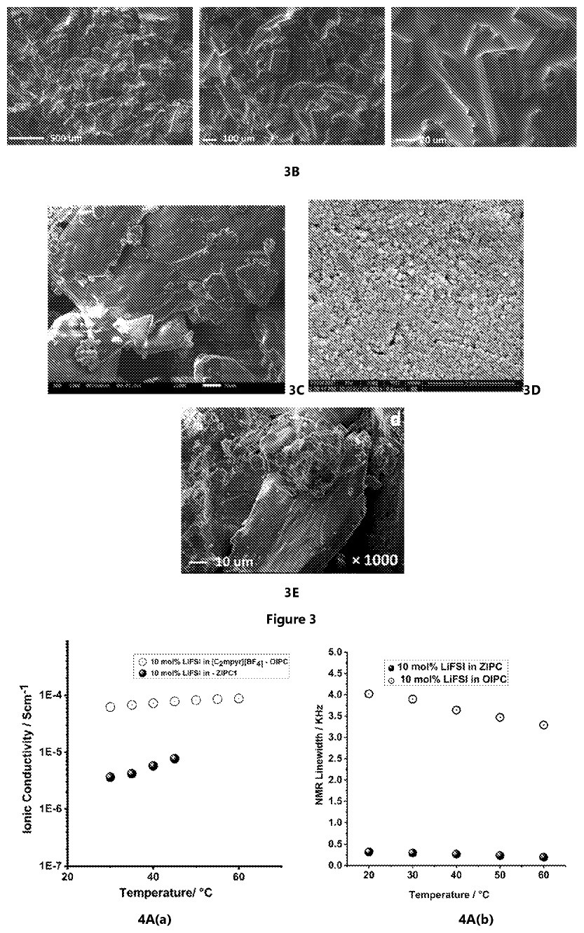

Examples of cations and anions of OIPCs which can be used as a starting point for design of the ZIPC compounds of the present invention are found in Trends in Chemistry, April 2019, Vol. 1, No. 1; J. Mater. Chem., 2010, 20, 2056-2062, and Phys. Chem. Chem. Phys., 2013, 15, 1339 (particularly FIG. 1, FIG. 2, FIG. 3 and Table 1), the entire contents of which describing cations and anions and OIPCs are incorporated herein by reference. Preferred examples of known OIPCs include [N_{1,1,1,1}][DCA], [C₂mpyr][FSI], [C₂mpyr][BF₄], [P_{1,2,2,2}][FSI], [P_{1,2,2,4}][PF₆], [P_{1,4,4,4}][FSI], [H₂im][Tf], [Hmim][Tf], [N_{2,2,3,3}][BBu₄], [N_{3,3,3,3}][BF₄], [C₂epyr][TFSI], [C₂epyr][FSI], [C₂epyr][PF₆], [C₂epyr][BF₄], [C₁mpyr][(FH)₂F] and [C₂mpyr][(FH)₂F], [C₄mpyr][TFSI], [(NH₂)₃][Tf], [2-Me-im][Tf], and [TAZm][PFBS].

Anion component for tethering—Suitably, at least one of the negatively charged functional groups carrying at least one negative charge may be derived from an anion from a known OIPC. Some preferred anions may be protic or aprotic anions, depending on the availability of labile proton(s). Some preferred anions may be di-anions or tri-anions. Some preferred anions may be symmetrical. Some preferred anions may be chiral.

Preferred anions that can be used for tethering may possess a ‘globular’ structure whereby the anion has a configurational shape presenting spherical symmetry around its centre by rotation around an axis. A further anion suitable for tethering in the ZIPC electrolyte composition of the invention may be one that has a diffuse or mobile negative charge which is able to reside or average across the anion structure when tethered in the ZIPC compound.

Suitably, one or more of the functional groups carrying a negative charge can be selected from the group of anions and particularly aprotic anions, consisting of: Tf, (FH)ₙF, where 1≤n≤3, and TFSI. Other suitable anions for forming the one or more of the functional groups carrying a negative charge can be selected from the group of anions consisting of: I, Br, PF₆, TFSI, BBu₄, CrO₃Cl, CrO₃Br, BF₄, FTFSI, DCA, FSI, and Tf. Centrosymmetric anions (e.g., hexafluorophosphate and tetrafluoroborate) are particularly preferred.

Desirable, at least one negatively charged functional group carrying at least one negative charge (F⁻) is derived from an anion, such as BF₄⁻, PF₆⁻, N(CN)₂, (CF₃SO₂)₂N⁻, (FSO₂)₂N⁻, OCN, SCN⁻, dicyanomethanide, carbamoyl cyano(nitroso)methanide, (C₂F₅SO₂)₂N⁻, (CF₃SO₂)₃C, C(CN)₃, B(CN)₄⁻, (C₂F₅)₃PF₃⁻, alkyl-SO₃⁻, perfluoroalkyl-SO₃⁻, aryl-SO₃⁻, I⁻, H₂PO₄⁻, HPO₄²⁻, sulfate, sulphite, nitrate, trifluoromethanesulfonate, p- toluenesulfonate, bis(oxalate)borate, acetate, formate, gallate, glycolate, BF₃(CN)⁻, BF₂(CN)₂⁻, BF(CN)₃⁻, BF₃(R)⁻, BF₂(R)₂⁻, BF(R)₃⁻ where R is an alkyl group (for example methyl, ethyl, propyl), cyclic sulfonyl amides, bis (salicylate)borate, perfluoroalkyltrifluoroborate, chloride, bromide, and transition metal complex anions (for example [Tb(hexafluoroacetylacetonate)₄]). Preferably, the anion is a fluorinated anion, for example, selected from the group consisting of: BF₄⁻, PF₆⁻, (CF₃SO₂)₂N⁻, (FSO₂)₂N⁻, BF₃(CN)⁻, BF₂(CN)₂⁻, BF(CN)₃⁻, BF₃(R)⁻, BF₂(R)₂⁻, BF(R)₃⁻ where R is an alkyl group (for example methyl, ethyl, propyl, butyl) (C₂F₅SO₂)₂N⁻. (C₂F₅)PF₃⁻, (C₂F₅PO₂)₂N, (CF₃SO₂)NCN, (CF₃SO₂)N(SO₂F), (CF₃CO)N(SO₂F) and perfluoroalq-SO₃⁻.

Other anions for tethering may be selected from the group consisting of the anions illustrated in FIG. 1C.

OIPC analogues—Examples of known OIPCs which can provide cations and anions for tethering together in the same molecule to form a ZIPC according to the invention, which include both protic and aprotic types, include N,N-methylethylpyrrolidinium tetrafluoroborate, N,N-methylpropylpyrrolidinium tetrafluoroborate, dimethylpyrrolidinium tetrafluoroborate, dimethylpyrrolidinium thiocyanate, N,N-ethylmethylpyrrolidinium thiocyanate, tetramethylammonium dicyanamide, tetraethylammonium dicyanamide, N,N-methylethylpyrrolidinium bis(trifluoromethanesulfonyl)amide, diethyl(methyl)isobutyl)phosphonium bis(fluorosulfonyl)amide, diethyl(methyl)(isobutyl)phosphonium tetrafluoroborate, diethyl(methyl)(isobutyl)phosphonium hexafluorophosphate, methy(triethyl)phosphonium bis(fluorosulfonyl)amide, methyl(triethyl)phosphonium bis(trifluoromethylsulfonyl)amide, triisobutyl(methyl)phosphonium hexafluorophosphate, triisobutyl(methyl)phosphonium bis(fluorosulfonyl)amide, triisobutyl(methyl)phosphonium tetrafluoroborate, triisobutyl(methyl)phosphonium thiocyanate, triethyl(methyl)phosphonium bis(fluorosulfonyl)imide, methylethyl pyrrolidium bis(fluorosulfonyl)amide, dimethylpyrrolidinium bis(fluorosulfonyl)amide, choline dihydrogen phosphate, choline trifluoromethanesulfonate, N-N-dimethylpropylenediammonium triflate, tri(isobutyl)phosphonium bis(trifluoromethanesulfonyl)amide, tri(isobutyl)phosphonium methanesulfonate, tri(isobutyl)phosphonium trifluoromethanesulfonate, tri(isobutyl)ammonium bis(trifluoromethanesulfonyl)amide, tri(isobutyl(phosphonium nitrate, tri(isobutyl)ammonium methanesulfonate, tri(isobutyl (ammonium trifluoromethanesulfonate, tri(isobutyl)ammonium nitrate, 1,2-bis[N-(N′-hexylimidazolium)ethane bis(hexafluorophosphate), and combinations thereof.
Preferred ZIPCs—A particularly preferred zwitterionic plastic crystal (ZIPC) compound has a structure as shown herein. Preferably, one or more of R′, R″ and R″′ are independently H, methyl, ethyl or propyl. Preferably each of R¹, R², and R³ are independently selected from H, methyl, ethyl or propyl, or halogen. Preferably Y is methyl, ethyl, or propyl. Preferably, L is methyl, ethyl, or propyl. Preferably, one or more of R′, R″ and R″' are independently is methyl, ethyl or propyl; each of R¹, R², R³ are F; Y is methyl and L is methyl. Preferred compounds include:

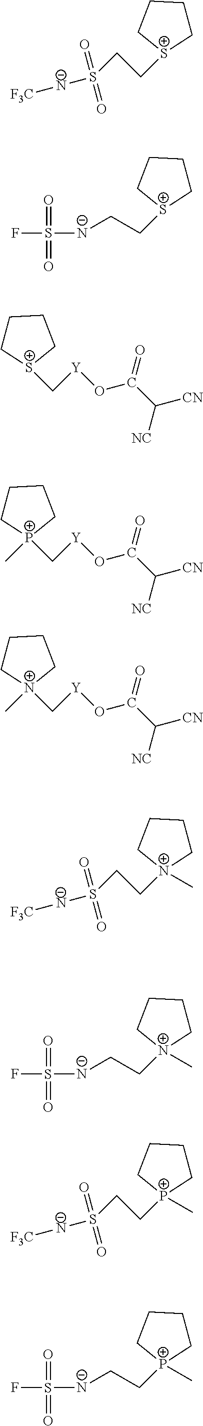

A particularly preferred zwitterionic plastic crystal (ZIPC) compound has one of the following general structures:

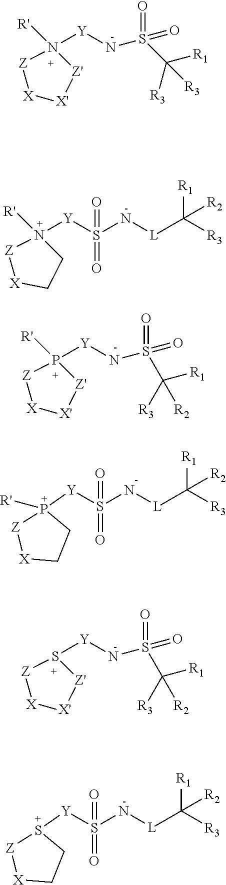

wherein: one or more of R′, R″ and R″′ are independently selected from H, or an optionally substituted C₁₋₆alkyl, an optionally substituted fluoroC₁₋₆alkyl or a halo group, or one of R′ and R″, R″ and R″′ or R′ and R″′ form an optionally substituted 5- or 6-membered saturated or unsaturated heterocyclic ring, each of R¹, R², and R³ are independently selected from H, an optionally substituted C₁₋₆ alkyl, optionally substituted fluoroC₁₋₆ alkyl, or a halo; Y is an optionally substituted C₁₋₆ alkyl; L is an optionally substituted C₁₋₆ alkyl; and independently each of Z and Z′ is O, S, NH, N, C₁₋₄alkyl; and independently each of X and X″ is O, S, NH, N, C, CH; and where present the ring is optionally substituted, wherein the optional substituents are selected from one or more of C₁₋₆alkyl, preferably methyl, ethyl or propyl, CN, OMe, OEt and CN. Preferably, R¹ is H, methyl, ethyl or propyl; each of R², R³, R⁴ are independently selected from H, methyl, ethyl or propyl, halogen; Y is methyl, ethyl, or propyl; L is methyl, ethyl, or propyl; and Z is methyl or ethyl; and X is O, S, NH, or CH. Preferably, R¹ is methyl, ethyl or propyl; each of R², R³, R⁴ are F; Y is methyl; L is methyl; and Z is methyl or ethyl; and X is O, S, NH, or CH.

A particularly preferred zwitterionic plastic crystal (ZIPC) compound has one of the following general structures:

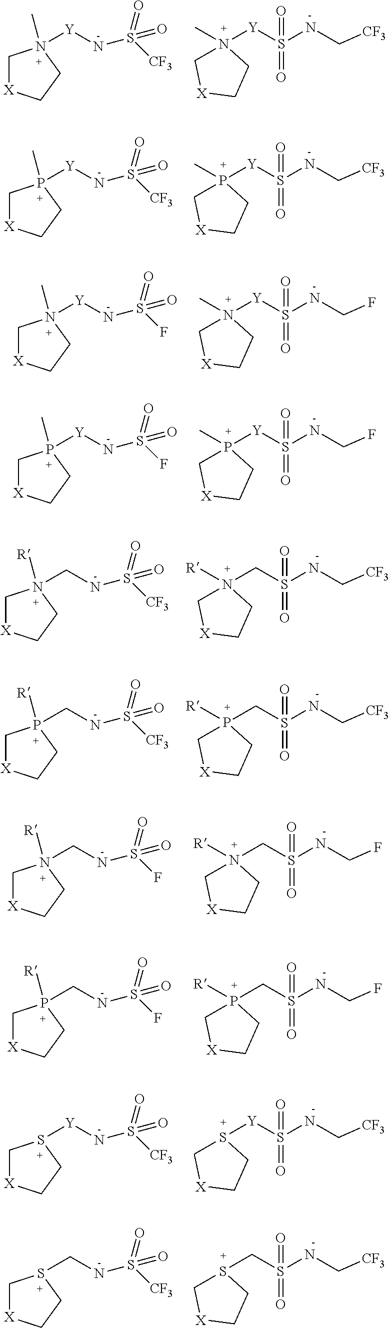

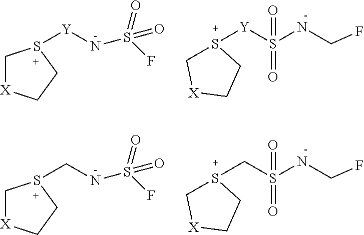

wherein: R′ is methyl, ethyl or propyl; each of R¹, R², R³ are F; Y is methyl; X is O, S, NH, or CH.

A preferred ZIPC compound has one of the following structures:

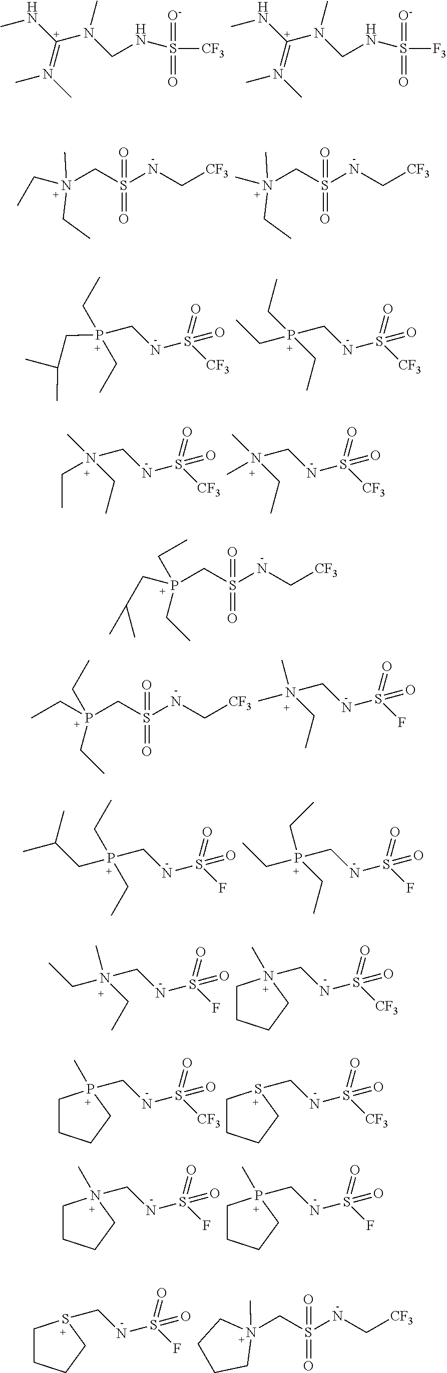

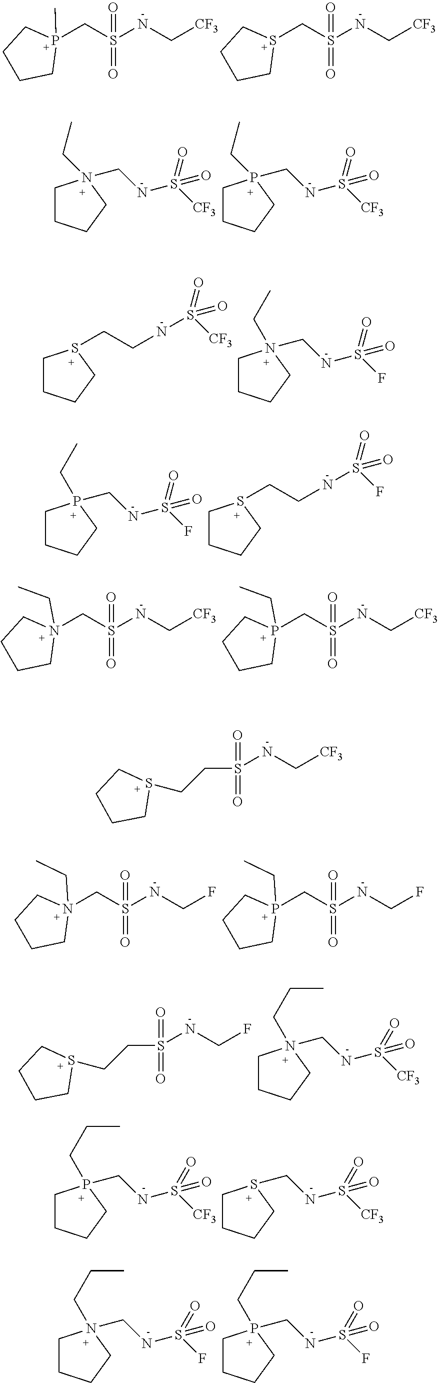

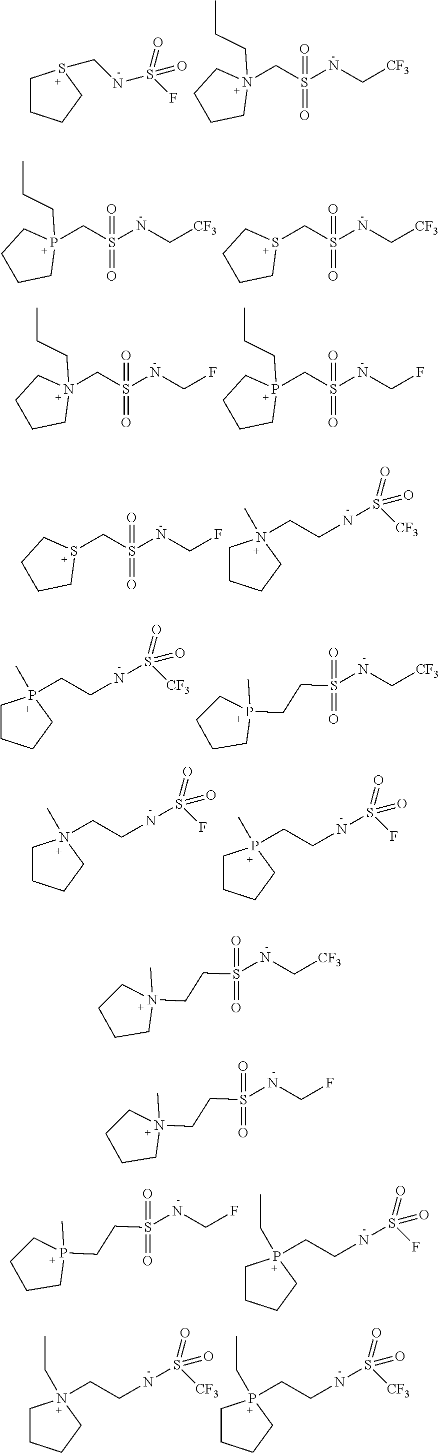

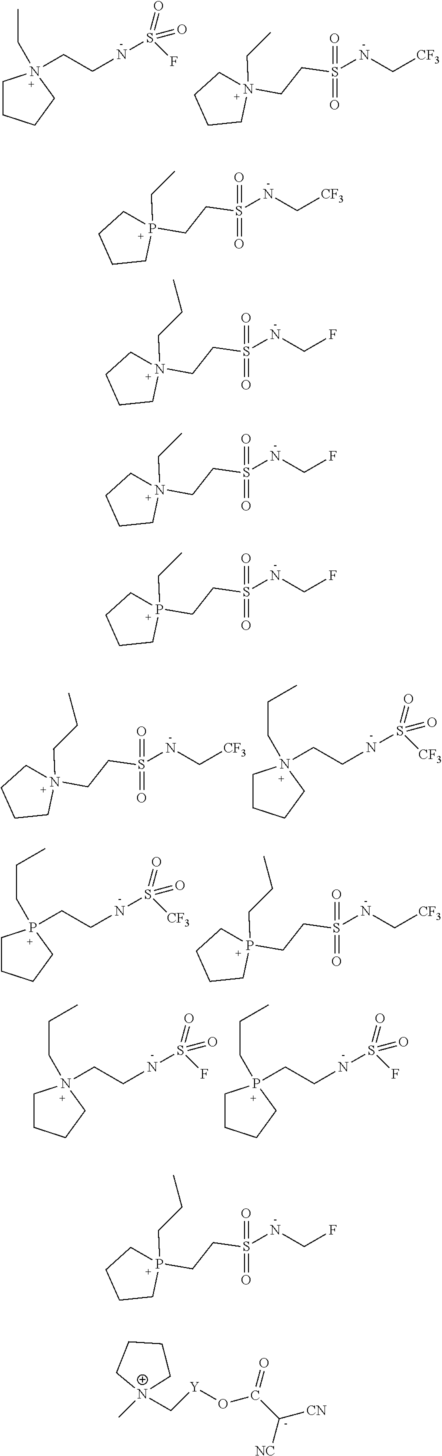

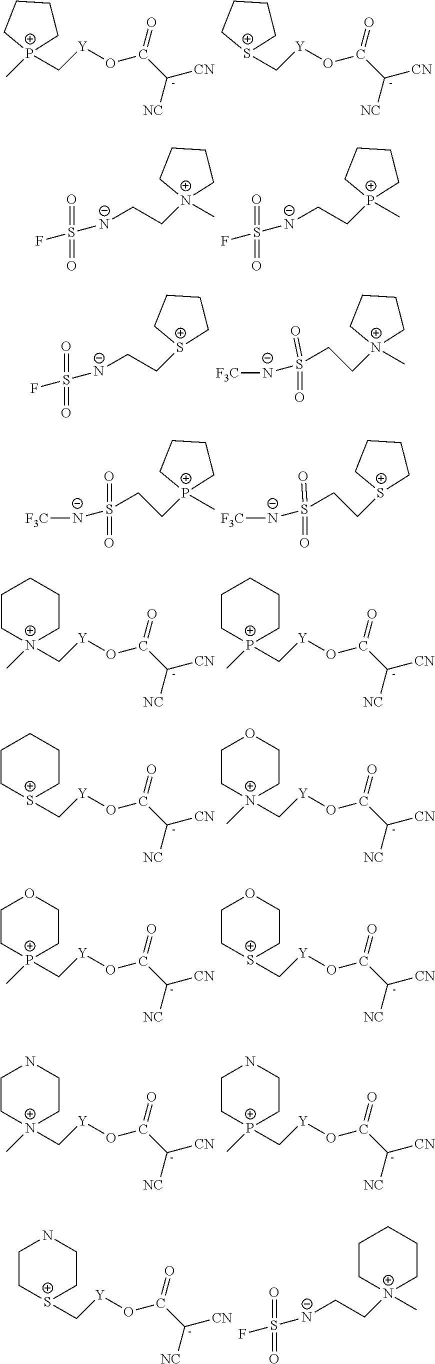

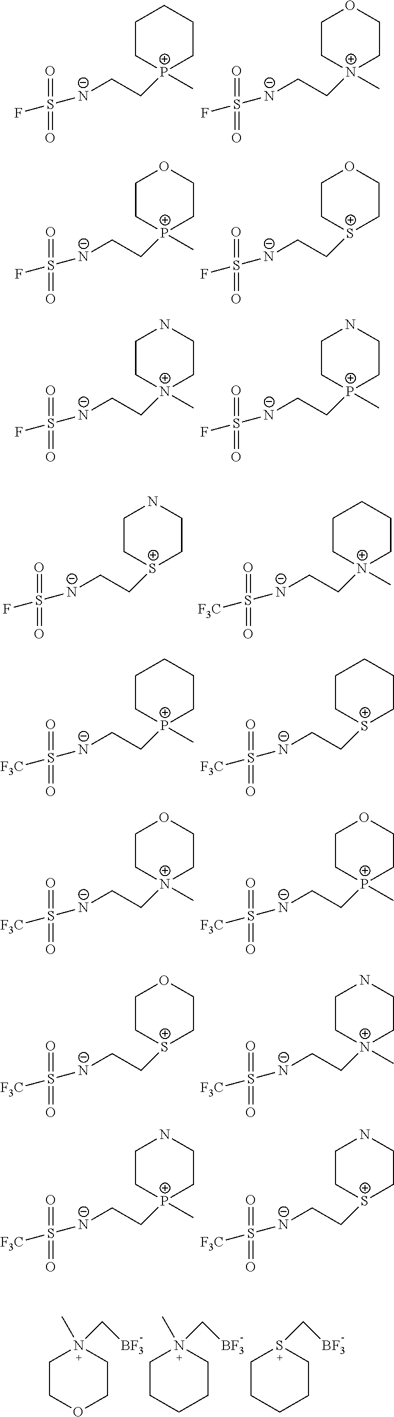

The ZIPCs of the invention may be used as solid state solvents.

Electrolyte composition/mixture—Also described are compositions comprising a zwitterionic plastic crystal (ZIPC) compound according the first aspect, doped with one or more of a salt, an acid, a base or a polymer typically used in electrolytes such as a polymer Li or Na functionalised polymer. Suitably such compositions may be solid state compositions or liquid compositions at room temperature, that is, depending on the amount of salt, the nature of the salt used as well as the nature of the ZIPC used. Solid state compositions are preferred at least where the ZIPC is used as the matrix material of the composition/electrolyte.

To be used as solid-state electrolytes (e.g., in batteries or fuel cells), target ions (e.g., Li⁺, Na⁺, or H⁺) must be incorporated into the ZIPC matrix to support charge/discharge processes. Doping even a small amount of ionic salt into the ZIPC matrix may significantly increase the ionic conductivity of the target ion in the ZIPC matrix. One explanation is that incorporating ion salts into the ZIPC creates additional vacancies/defects, leading to a higher concentration of diffusive ions, and therefore higher conductivities. An alternative mechanism is that a liquid phase with a mixed (Li salt and ZIPC) composition is present at the grain boundaries of otherwise mostly bulk ZIPC.

Preferably, the composition comprises ZIPC and at least one ionic salt, wherein the salt is present in a concentration of at least about 5 mol %. Suitably, the ionic salt is present in a concentration of at least about 5 mol %, at least about 10 mol %, at least about 15 mol %, at least about 20 mol %, at least about 25 mol %, at least about 30 mol %, at least about 35 mol %, at least about 40 mol %, at least about 45 mol %, at least about 50 mol %, at least about 55 mol %, at least about 60 mol %, at least about 65 mol %, at least about 70 mol %, at least about 75 mol %, at least about 80 mol %, at least about 85 mol %, at least about 90 mol %, at least about 95 mol %.

Suitably, the ionic salt is one or more of an alkali metal, alkaline earth, or transition metal salt. Preferred ionic salts include Li, Na, K, Ca, Al, Mg, Zn salts. Suitably anions for these salts include bis(trifluoromethanesulfonyl)imide, TFSI; bis(fluorosulfonyl)imide, FSI; fluorosulfonyl(trifluoro-methanesulfonyl)imide, FTFSI; trifluoromethane-sulfonate; tetrafluoro-borate, BF₄; perfluorobutane-sulfonate, PFBS; hexafluorophosphate, PF₆; Tetracyanoborate, B(CN)₄; dicyanamide, DCA; thiocyanate, SCN; cyclic perfluoro-sulfonylamide, CPFSA, and carboranes.

Desirably, the ionic salt is a lithium salt, for example, selected from the group consisting of: LiBF₄, LiFSI, Lithium bis(trifluoromethanesulfonyl)imide (Li[TFSI]), lithium (bis(fluorosulfonyl)imide (Li[FSI]), lithium triflate (Li[OTf]), lithium perchlorate (LiClO₄), lithium dicyanamide (LiDCA), lithium cyanate (LiOCN), lithium thiocyanate (LiSCN), lithium bis[(pentafluoro-ethyl)sulfonyl]imide, lithium 2,2,2-trifluoromethylsulfonyl-N-cyanoamide (TFSAM), lithium 2,2,2-trifluoro-N-(trifluoromethylsulfonyl) acetamide (TSAC), lithium nonafluorobutanesulfonate (NF), lithium carborane, lithium difluoro(oxolato)borate and combinations thereof.

Preferably, the doped salt is a Li salt, such as LiNTf₂, wherein the ZIPC composition has a transference number of greater than 0.4 as determined by electrochemically or by NMR. Such techniques are known in the art. One example of an electrochemical method for ion transference number is the Bruce Vincent method which is well known in the art.

Desirably, the ionic salt is a sodium salt, for example, selected from the group consisting of: NaBF₄, NaFSI, sodium bis(trifluoromethanesulfonyl)imide (Na[TFSI]), sodium(bis(fluorosulfonyl)imide (Na[FSI]), sodium triflate (Na[OTf]), sodium perchlorate (NaClO₄), sodium dicyanamide (NaDCA), sodium cyanate (NaOCN), sodium thiocyanate (NaSCN), lithium bis[(pentafluoro-ethyl)sulfonyl]imide, sodium 2,2,2-trifluoromethylsulfonyl-N-cyanoamide (TFSAM), sodium 2,2,2-trifluoro-N-(trifluoromethylsulfonyl) acetamide (NaTSAC), lithium nonafluorobutanesulfonate (NaNF), sodium carborane, sodium difluoro(oxolato)borate and combinations thereof. Particularly preferred Na salts include sodium bis(trifluoromethanesulfonyl)imide (Na[TFSI]), sodium (bis(fluorosulfonyl)imide (Na[FSI]), sodium triflate (NaOTf), sodium perchlorate (NaClO₄), sodium dicyanamide (NaDCA), sodium cyanate (NaOCN) sodium tetrafluoroborate (NaBF₄), sodium hexafluorophosphate (NaPF₆), and combinations thereof.

Desirably, the ionic salt is an iodide salt selected from the group consisting of: AgI, NaI, KI, guanidinium iodide, Nme₄I, N(Pr)₄I, N(Et)₄I and combinations thereof. The iodide salt is typically provided in combination with iodine such that the combination dissociates into an I⁻/I₃⁻ couple.

Desirably, the ZIPC composition is doped with acid or base. Incorporating excess acid or base into protic ZIPCs facilitates high proton conductivity. It is thought that protons are primarily transported through a percolated grain boundary phase.

Preferably, the ZIPC composition comprises a ZIPC compound and acid, wherein the acid is present in a concentration of at least about 5 mol %. Suitably, the acid is present in a concentration of at least about 5 mol %, at least about 10 mol %, at least about 15 mol %, at least about 20 mol %, at least about 25 mol %, at least about 30 mol %, at least about 35 mol %, at least about 40 mol %, at least about 45 mol %, at least about 50 mol %, at least about 55 mol %, at least about 60 mol %, at least about 65 mol %, at least about 70 mol %, at least about 75 mol %, at least about 80 mol %, at least about 85 mol %, at least about 90 mol %, at least about 95 mol %. Suitable acids include triflic acid, bis(trifluoromethanesulfonyl)amine, methanesulfonic acid, sulfuric acid, phosphoric acid, nitric acid, formic acid, tetrafluoroboric acid.

Preferably, the ZIPC composition comprises a ZIPC compound and base, wherein the base is present in a concentration of at least about 5 mol %. Suitably, the base is present in a concentration of at least about 5 mol %, at least about 10 mol %, at least about 15 mol %, at least about 20 mol %, at least about 25 mol %, at least about 30 mol %, at least about 35 mol %, at least about 40 mol %, at least about 45 mol %, at least about 50 mol %, at least about 55 mol %, at least about 60 mol %, at least about 65 mol %, at least about 70 mol %, at least about 75 mol %, at least about 80 mol %, at least about 85 mol %, at least about 90 mol %, at least about 95 mol %. Suitable bases include imidazole, methylamine, ethylamine, propylamine, butylamine, tert-butylamine, 2-methoxyethylamine, 3-methoxypropyla mine di methyl amine, diethylamine, di butylamine, N-methylbutylamine, N-ethylbutylamine trimethylamine, triethylamine tributylamine, N, N-dimethylethylamine aniline 2-fluoropyridine, 1-methylimidazole or 1,2-dimethylimidazole. Preferred bases include imidazole.

Preferably, the solid-state composition further comprises one or more additional components selected from polymers, particularly lithium or sodium functionalised polymers, binders such as PVDF, ionomers, dendrimers, and inorganic fillers to form tertiary composites. In one embodiment, the solid-state composition may be provided in the form of a membrane.

Preferred compounds, when doped with ionic salts such as alkali metal, alkaline earth, or transition metal ions, exhibit an ion transference number of greater than 0.4, as determined electrochemically or NMR. More preferably, the ion transference number is greater than 0.4, greater than 0.45, greater than 0.5, greater than 0.55, greater than 0.6, greater than 0.65, greater than 0.7, greater than 0.75, greater than 0.8, greater than 0.85, greater than 0.9, greater than 0.95 as determined electrochemically or NMR.

Preferred ZIPC compounds, when doped with lithium ions or sodium ions, exhibit a lithium ion transference number of greater than 0.4, determined electrochemically or by NMR. More preferred ZIPC compounds, when doped with lithium ions or sodium, exhibit an ion transference number of greater than 0.5, 0.6, 0.7, 0.8, or 0.9. Most preferred ZIPC compounds, when doped with lithium ions or sodium ions, exhibit an ion transference number of approximately 1.

Preferred ZIPC compounds, when doped with a lithium salt to form a mixture exhibit a lithium diffusion coefficient in the range of 10⁻¹³ to 10⁻¹⁰, m²s⁻¹, preferably 10⁻¹³ to 10⁻⁸, m²s⁻¹, more preferably 10⁻¹³ to 10⁻⁶ m²s⁻¹, as measured by NMR at 25° C. A preferred mixture of a ZIPC compound and a lithium salt, exhibits a lithium self diffusion coefficient of at least 10⁻¹³ m²s⁻¹ as measured by NMR at 25° C. A most preferred mixture of a ZIPC compound and a lithium salt, exhibits a lithium self diffusion coefficient at least 10⁻⁶ m²s⁻¹ as measured by NMR at 25° C.

The ZIPC compound and/or the electrolyte composition comprising the ZIPC compound and at least ionic salt is preferably a solid, preferably up to at least 80° C. and preferably over a wide concentration range of ionic salt while maintaining high ionic conductivity.

The electrolyte compositions of the present invention advantageously offer high ionic conductivity at lower temperature relative to most polymer electrolytes. As a result, electrochemical cells based on the electrolytes of the may operate at lower temperatures relative to conventional solid-state cells.

The electrolyte composition of the invention can advantageously present as a solid up to a desired temperature over a wide range of ionic salt concentrations. Preferably, the ZIPC compound and/or the electrolyte comprising a matrix of doped ZIPC compound presents as a solid up to at least 30° C., at least 40° C., at least 50° C., at least 60° C., at least 70° C., at least 80° C., at least 90° C., at least 100° C., at least 110° C., at least 120° C., at least 130° C., at least 140° C., at least 150° C., at least 160° C., at least 170° C., at least 180° C., at least 190° C., at least 200° C., at least 210° C., at least 220° C., at least 230° C., at least 240° C., or at least 250° C.

In some embodiments, the ZIPC and/or the electrolyte composition of the invention is solid throughout the entire composition meaning the entire volume of the electrolyte composition is in the solid state. However, provided the ZIPC and/or the electrolyte presents as a solid, a fraction of the matrix/composition may nevertheless be in the liquid phase. There is no limitation as to the extent of the fraction of matrix/composition that is in the liquid phase, provided the material/composite presents as a solid up to a desired temperature. Those skilled in the art would be capable to determine suitable values of volume fraction that is in the liquid phase for a given material on the basis of the phase diagram of the material.

In some embodiments, the temperature at which the electrolyte composition of the invention may present a volume fraction which is in the liquid phase is up to at least 30° C., at least 40° C., at least 50° C., at least 60° C., at least 70° C., at least 80° C., at least 90° C., at least 100° C., at least 110° C., at least 120° C., at least 130° C., at least 140° C., at least 150° C., at least 160° C., at least 170° C., at least 180° C., at least 190° C., at least 200° C., at least 210° C., at least 25 220° C., at least 230° C., at least 240° C., at least 250° C., at least 300° C., or at least 350° C.

There is no particular limitation as to the ionic salt concentration in the solid state ZIPC composition of the invention. However, preferably the composition presents as a solid up to at least 50° C. In some embodiments, the ionic is present at a concentration of at least 5 mol %, at least 10 mol %, at least 15 mol %, at least 20 mol %, at least 25 mol %, at least 30 mol %, at least 35 mol %, at least 40 mol %, at least 45 mol %, at least 50 mol %, at least 55 mol %, at least 60 mol %, at least 65 mol %, at least 70 mol %, at least 75 mol %, at least 80 mol %, at least 85 mol %, at least 90 mol %, or at least 95 mol %, relative to the total moles of ionic salt and ZIPC compound combined.

Preferred electrolyte compositions of the invention have an ionic conductivity of at least 10⁻⁹ S/cm when in sub-melting phase. In some embodiments, the ionic conductivity of the electrolyte composition is at least 10⁻⁹S/cm, at least 10⁻⁸ S/cm, at least 10⁻⁷ S/cm, at least 10⁻⁶ S/cm, at least 10⁻⁵ S/cm, at least 10⁻⁴ S/cm, at least 10⁻³ S/cm at room temperature as determined by electrochemical impedance spectroscopy (EIS).

Electrochemical cells and application—Described herein is a use of a ZIPC compound/matrix or a ZIPC composition in an application requiring ion conduction, including an electrochemical device such as a fuel cell, an energy storage device, a supercapacitor or a dye-sensitised solar cell. Described herein is a use of ZIPC composition in an application requiring ion conduction, including an electrochemical device such as a fuel cell, an energy storage device, a supercapacitor or a dye-sensitised solar cell.

Described herein is an electrolyte comprising one or more ZIPC compounds of the invention as a matrix or as an additive for an electrolyte and/or one or more ZIPC compositions/composites according to the invention as an electrolyte. Preferably, the ZIPC of the invention may be used in an electrochemical cell as an electrolyte matrix or in an electrolyte material as an additive. The electrolyte may be a solid state electrolyte or a liquid electrolyte, for example, at room temperature.

Preferably, the electrochemical cell or device is an energy storage device such as a Na battery or a Li battery, particularly a rechargeable or secondary battery. The materials described herein are particularly suited to cells involving high voltage chemistries, for example, over 4.5 V vs Li/Li⁺. Described herein is a fuel cell device comprising a zwitterionic plastic crystal (ZIPC) electrolyte matrix, optionally doped with an acid, base or salt dopant. Described herein is a use of a zwitterionic plastic crystal (ZIPC) in a protonated form in an application requiring proton conduction including a fuel cell. A base doped ZIPC composition may be used as an anhydrous proton conductor, preferably wherein the base is imidazole.

Desirably, the present invention provides an energy storage device comprising a negative electrode, a positive electrode, and an electrolyte comprising a ZIPC compound as matrix or an additive or a ZIPC electrolyte composition/composite according to the invention.

Definitions—As used herein, the term ‘alkyl’ describes a group composed of at least one carbon and hydrogen atom, and denotes straight chain, branched or cyclic alkyl, for example C₁₋₂₀ alkyl, e.g. C₁₋₁₀ or C₁₋₆. Examples of straight chain and branched alkyl include methyl, ethyl, n-propyl, isopropyl, n-butyl, sec-butyl, t-butyl, n-pentyl, 1,2-dimethylpropyl, 1,1-dimethyl-propyl, hexyl, 4-methyl pentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethyl pentyl, 1,2,3-trimethylbutyl, 1,1,2-trimethylbutyl, 1,1,3-trimethylbutyl, octyl, 6-methylheptyl, 1-methylheptyl, 1,1,3,3-tetramethylbutyl, nonyl, 1-, 2-, 3-, 4-, 5-, 6- or 7-methyloctyl, 1-, 2-, 3-, 4- or 5-ethylheptyl, 1-, 2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecy I, 1-, 2-, 3-, 4-, 5- or 6-propylnonyl, 1-, 2-, 3- or 4-butyloctyl, 1-2-pentylheptyl and the like. Examples of cyclic alkyl include mono- or polycyclic alkyl groups such as cyclopropyl, cyclobutyl, cyclopentyl, cyclohexyl, cycloheptyl, cyclooctyl, cyclononyl, cyclodecyl and the like. Where an alkyl group is referred to generally as ‘propyl’, butyl' etc, it will be understood that this can refer to any of straight, branched and cyclic isomers where appropriate. An alkyl group may be optionally substituted by one or more substituents, which include substituents in which a carbon has been substituted with a heteroatom (such as O, N, S), as herein defined.

Examples of optional substituents include alkyl, (e.g. C₁₋₆alkyl such as methyl, ethyl, propyl, butyl, cyclopropyl, cyclobutyl, cyclopentyl or cyclohexyl), hydroxyalkyl (e.g. hydroxymethyl, hydroxyethyl, hydroxypropyl), alkoxyalkyl (e.g. methoxymethyl, methoxyethyl, methoxypropyl, ethoxymethyl, ethoxyethyl, ethoxypropyl etc) alkoxy (e.g. C₁₋₆alkoxy such as methoxy, ethoxy, propoxy, butoxy, cyclopropoxy, cyclobutoxy), halo, trifluoromethyl, trichloromethyl, tribromomethyl, hydroxy, phenyl (which itself may be further substituted e.g., by C₁₋₆alkyl, halo, hydroxy, hydroxyC₁₋₆ alkyl, C₁₋₆ alkoxy, haloC₁₋₆alkyl, cyano, nitro OC(O)C₁₋₆alkyl, and amino), benzyl (wherein benzyl itself may be further substituted e.g., by C₁₋₆alkyl, halo, hydroxy, hydroxyC₁₋₆alkyl, C₁₋₆alkoxy, haloC₁₋₆alkyl, cyano, nitro OC(O)C₁₋₆alkyl, and amino), phenoxy (wherein phenyl itself may be further substituted e.g., by C₁₋₆alkyl, halo, hydroxy, hydroxyC₁₋₆alkyl, C₁₋₆alkoxy, haloC₁₋₆alkyl, cyano, nitro OC(O)C₁₋₆alkyl, and amino), benzyloxy (wherein benzyl itself may be further substituted e.g., by C₁₋₆alkyl, halo, hydroxy, hydroxyC₁₋₆alkyl, C₁₋₆ alkoxy, haloC₁₋₆alkyl, cyano, nitro OC(O)C₁₋₆ alkyl, and amino), amino, alkylamino (e.g. C₁₋₆alkyl, such as methylamino, ethylamino, propylamino etc), dialkylamino (e.g. C₁₋₆alkyl, such as dimethylamino, diethylamino, dipropylamino), acylamino (e.g. NHC(O)CH3), phenylamino (wherein phenyl itself may be further substituted e.g., by C₁₋₆alkyl, halo, hydroxy, hydroxyC₁₋₆alkyl, C₁₋₆alkoxy, haloC₁₋₆alkyl, cyano, nitro OC(O)C₁₋₆alkyl, and amino), nitro, formyl, —C(O)-alkyl (e.g. C₁₋₆alkyl, such as acetyl), O—C(O)-alkyl (e.g. C₁₋₆ alkyl, such as acetyloxy), benzoyl (wherein the phenyl group itself may be further substituted e.g., by C₁₋₆alkyl, halo, hydroxy hydroxyC₁₋₆alkyl, C₁₋₆ alkoxy, haloC₁₋₆alkyl, cyano, nitro OC(O)C₁₋₆alkyl, and amino), replacement of CH₂ with C═O, CO₂H, CO₂alkyl (e.g. C₁₋₆alkyl such as methyl ester, ethyl ester, propyl ester, butyl ester), CO2phenyl (wherein phenyl itself may be further substituted e.g., by C₁₋₆alkyl, halo, hydroxy, hydroxyl C₁₋₆alkyl, C₁₋₆alkoxy, halo C₁₋₆alkyl, cyano, nitro OC(O)C₁₋₆alkyl, and amino), CONH₂, CONHphenyl (wherein phenyl itself may be further substituted e.g., by C₁₋₆alkyl, halo, hydroxy, hydroxyl C₁₋₆alkyl, C₁₋₆ alkoxy, halo C₁₋₆alkyl, cyano, nitro OC(O)C₁₋₆alkyl, and amino), CONHbenzyl (wherein benzyl itself may be further substituted e.g., by C₁₋₆alkyl, halo, hydroxy hydroxyl C₁₋₆alkyl, C₁₋₆alkoxy, halo C₁₋₆alkyl, cyano, nitro OC(O)C₁₋₆alkyl, and amino), CONHalkyl (e.g. C₁₋₆alkyl such as methyl ester, ethyl ester, propyl ester, butyl amide) CONHdialkyl (e.g. C₁₋₆alkyl) aminoalkyl (e.g., HN C₁₋₆alkyl-, C₁₋₆alkylHN—C₁₋₆alkyl- and (C₁₋₆alkyl)₂N—C₁₋₆allyl-),thioalkyl (e.g., HS C₁₋₆alkyl-), carboxyalkyl (e.g., HO₂CC₁₋₆alkyl-), carboxyesterallyl (e.g., C₁₋₆ alkylO₂CC₁₋₆allyl-), amidoalkyl (e.g., H₂N(O)CC₁₋₆allyl-, H(C₁₋₆alkyl)N(O)CC₁₋₆alkyl-), formylalkyl (e.g., OHCC₁₋₆allyl-), acylalkyl (e.g., C₁₋₆alkyl(O)CC₁₋₆alkyl-), nitroalkyl (e.g., O₂NC₁₋₆allyl-), sulfoxidealkyl (e.g., R^{f}(O)SC₁₋₆allyl where R^{f} is as herein as defined for example alkyl, such as C₁₋₆alkyl(O)SC₁₋₆alkyl-), sulfonylalkyl (e.g., Rf(O)₂SC₁₋₆alkyl where R^{f} is as herein defined for example alkyl, such as C₁₋₆allyl(O)₂SC₁₋₆alkyl-), sulfonamidoalkyl (e.g., ₂HR^{f}N(O)SC₁₋₆alkyl where R^{f} is as herein defined, for example alkyl, such as H(C₁₋₆allyl)N(O)SC₁₋₆allyl-).

The term ‘halogen’ (‘halo’) denotes fluorine, chlorine, bromine or iodine (fluoro, chloro, bromo or iodo). Preferred halogens are chlorine, bromine or iodine.

The heterocyclyl group may be saturated or partially unsaturated, i.e. possess one or more double bonds. Particularly preferred heterocyclyl are 5-6 and 9-10 membered heterocyclyl. Suitable examples of heterocyclyl groups may include azridinyl, oxiranyl, thiiranyl, azetidinyl, oxetanyl, thietanyl, 2H-pyrrolyl, pyrrolidinyl, pyrrolinyl, piperidyl, piperazinyl, morpholinyl, indolinyl, imidazolidinyl, imidazolinyl, pyrazolidinyl, thiomorpholinyl, dioxanyl, tetrahydrofuranyl, tetrahydropyranyl, tetrahydropyrrolyl, tetrahydrothiophenyl, pyrazolinyl, dioxalanyl, thiazolidinyl, isoxazolidinyl, dihydropyranyl, oxazinyl, thiazinyl, thiomorpholinyl, oxathianyl, dithianyl, trioxanyl, thiadiazinyl, dithiazinyl, trithianyl, azepinyl, oxepinyl, thiepinyl, indenyl, indanyl, 3H-indolyl, isoindolinyl, 4H-quinolazinyl, chromenyl, chromanyl, isochromanyl, pyranyl and dihydropyranyl. A heterocyclyl group may be optionally substituted by one or more optional substituents as herein defined. The term ‘heterocyclylene’ is intended to denote the divalent form of heterocyclyl.

The term ‘heteroaryl’ includes any of monocyclic, polycyclic, fused or conjugated hydrocarbon residues, wherein one or more carbon atoms are replaced by a heteroatom so as to provide an aromatic residue. Preferred heteroaryl have 3-20 ring atoms, e.g. 3-10. Particularly preferred heteroaryl are 5-6 and 9-10 membered bicyclic ring systems. Suitable heteroatoms include, O, N, S, P and Se, particularly O, N and S. Where two or more carbon atoms are replaced, this may be by two or more of the same heteroatom or by different heteroatoms. Suitable examples of heteroaryl groups may include pyridyl, pyrrolyl, thienyl, imidazolyl, furanyl, benzothienyl, isobenzothienyl, benzofuranyl, isobenzofuranyl, indolyl, isoindolyl, pyrazolyl, pyrazinyl, pyrimidinyl, pyridazinyl, indolizinyl, quinolyl, isoquinolyl, phthalazinyl, 1,5-naphthyridinyl, quinozalinyl, quinazolinyl, quinolinyl, oxazolyl, thiazolyl, isothiazolyl, isoxazolyl, triazolyl, oxadialzolyl, oxatriazolyl, triazinyl, and furazanyl. A heteroaryl group may be optionally substituted by one or more optional substituents as herein defined. The term ‘heteroarylene’ is intended to denote the divalent form of heteroaryl.

The term ‘sulfoxide’, either alone or in a compound word, refers to a group R^{f}—S(O)R^{f} wherein Rf is selected from hydrogen, alkyl, alkenyl, alkynyl, aryl, heteroaryl, heterocyclyl, carbocyclyl, and aralkyl. Examples of preferred R^{f} include C₁₋₂₀alkyl, preferably C₁₋₆alkyl, most preferably C₁₋₃alkyl, phenyl and benzyl.

The term ‘sulfonyl’, either alone or in a compound word, refers to a group S(O)₂—R^{f}, wherein R^{f} is selected from hydrogen, halides, alkyl, alkenyl, alkynyl, aryl, heteroaryl, heterocyclyl, carbocyclyl and aralkyl. Examples of preferred Rf include C₁₋₂₀alkyl, phenyl and benzyl.

The term ‘sulfonamide’, either alone or in a compound word, refers to a group S(O)NR^{f}R^{f} wherein each Rf is independently selected from hydrogen, alkyl, alkenyl, alkynyl, aryl, heteroaryl, heterocyclyl, carbocyclyl, and aralkyl. Examples of preferred R^{f} include C₁₋₂₀alkyl, phenyl and benzyl. In a preferred embodiment at least one R^{f} is hydrogen. In another form, both R^{f} are hydrogen.

The term ‘heteroatom’ or ‘hetero’ as used herein in its broadest sense refers to any atom other than a carbon atom which may be a member of a cyclic organic group. Particular examples of heteroatoms include nitrogen, oxygen, sulfur, phosphorous, boron, silicon, selenium and tellurium, more particularly nitrogen, oxygen and sulfur.

The present invention is described with reference to the following examples. It is to be understood that the examples are illustrative of and not limiting to the invention described herein.

The physical, thermal and electrochemical properties of one of the compounds, ZIPC1 (with a BF₃⁻ charge, compound 1 in FIG. 1) has been compared to the analogous OIPC (i.e. with separate cation and anion) to probe the advantages of tethering the ion species. This species was chosen as the analogous OIPC [C₂mpyr][BF₄] and [C₂mpyr][NTf₂] (FIG. 1) whose efficacy as electrolytes for lithium metal batteries has been effectively demonstrated.

Use of the more charge diffuse and hydrolytically stable sulfonylimide groups, instead of the BF₃⁻ used in the first materials, was predicted to be even more beneficial for decreasing coordination with Li or Na salts and to make the material more disordered. Other cations can be used within the zwitterion plastic crystals, in particular morpholinium and piperidinium moieties (FIG. 1), ethyl-substituted pyrrolidiniums to make the analogous structures of 8 and 9 (FIG. 1), with the fluorosulfonylimide group.

EXAMPLES—Aprotic ZIPCs and protic ZIPCs—A number of examples of zwitterionic plastic crystals (ZIPCs) are shown with comparison of analogous established OIPCs in FIG. 1. For use as proton conductors, the ZIPC compounds facilitate the conduction of the H⁺ ion (proton) while themselves remaining immobile as a matrix material. They are doped with either an acid or a base. Base doping is preferred as being more effective in terms of proton conduction.

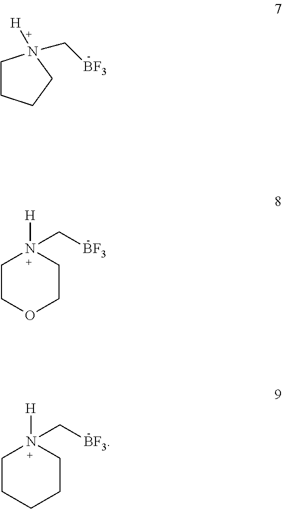

To investigate the efficacy of these materials as electrolytes for lithium batteries, the zwitterion ZIPC1 was combined with the lithium salts, LiFSI or LiBF₄ in the form of a salt doped ZIPC composition. The salt doped ZIPC compositions were investigated at both 10 mol % (Example 1) and 90 mol % (Example 2) concentration. Although the high lithium salt content introduces a large amount of free anions (FSI or BF₄) that compete with the transference number of the Li cation, a high concentration of Li ion can be very advantageous for device performance (e.g., by decreasing polarisation) and it is believed that ZIPC1 helps dissociate Li⁺ from the respective counter anion to result in increases in lithium ion transport compared to the equivalent high Li salt content OIPC electrolyte. This demonstrates the general benefits of adding a ZIPC, even when used as a small proportion of a conventional electrolyte mixture as an additive to facilitate target ion dissociation. The ionic conductivity of pure ZIPC1 and [C₂mpyr][BF₄] OIPC and their equivalent mixtures with LiFSI as a function of temperature was investigated and is reported below.

Proof of concept as electrolyte—Preliminary DSC, NMR, conductivity, cell cycling and transference number for many of these electrolytes are available. Test and results are described in more detail below. In particular, the inventors have investigated the following electrolytes:

10 mol % LiFSI doped ZIPC1 electrolyte (Example 1): This is compared to 10 mol % LiFSI in the analogous [C2mpyr][BF4] OIPC, which has been previously studied extensively in prior work in the research group of the inventors to further demonstrate the advantages of tethering the cation and anion.

90 mol % LiFSI doped ZIPC electrolyte (Example 2): This is compared to the neat ZIPC. This high Li salt concentration is likely to give good battery performance.

Protic ZIPC electrolytes (Example 3):—To investigate the benefits of protic ZIPCs as anhydrous proton conductors, ZIPCs have been doped with either acid or base.

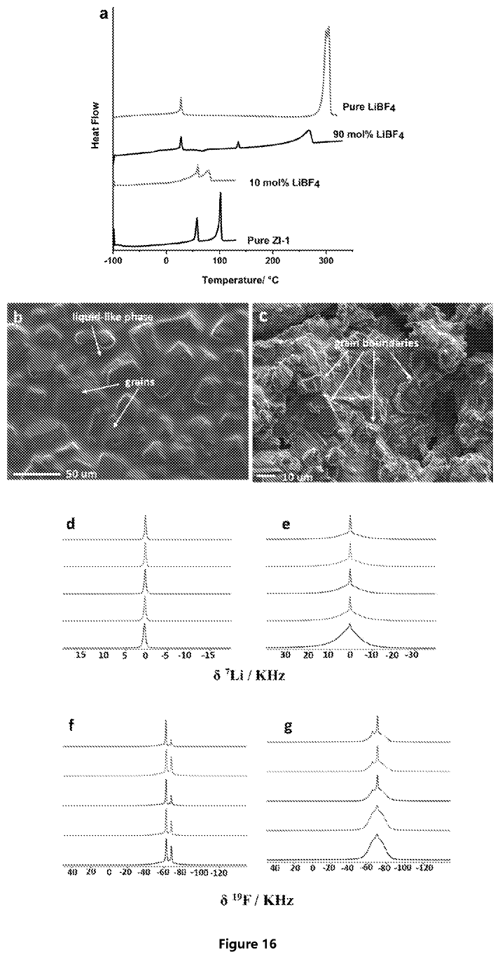

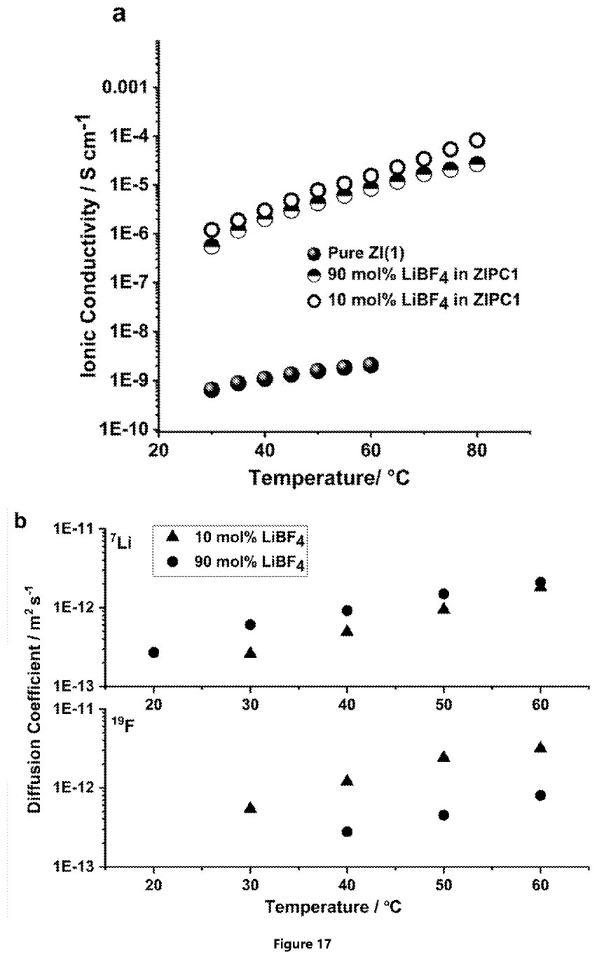

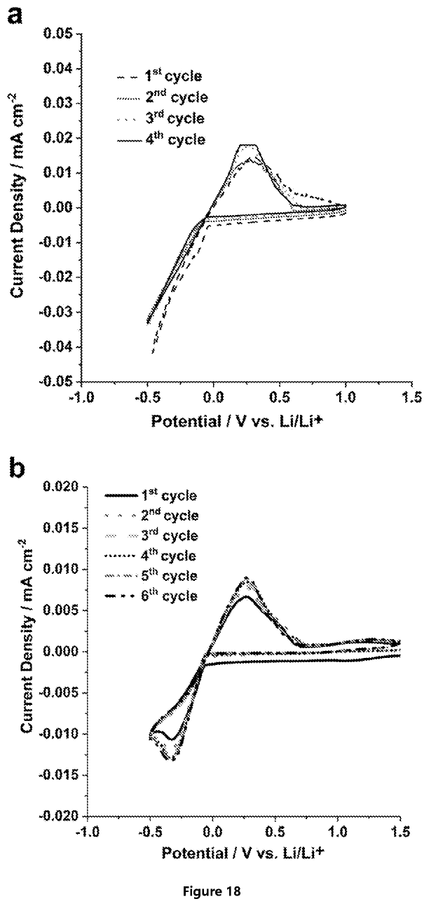

ZIPC1 and its mixtures with 10 and 90 mol % LiBF₄ (Example 4):—DSC analysis, FIG. 16a, shows that the melting transition temperature and the entropy of fusion of the 10 mol % LiBF₄ in ZIPC1 is depressed compared to the pure ZIPC1. This effect has also been observed in mixtures of other plastic crystals with Li or Na salts, attributed to formation of either a eutectic composition or the creation of more defects. Upon increasing the LiBF₄ concentration to 90 mol %, a phase with higher melting point (220° C.) and low entropy of fusion (4.9 J/molK) is formed. The SEM (FIG. 16b) shows the morphology of 10 mol % LiBF₄ in ZIPC1, with grains connected by an amorphous or liquid-like phase that may create pathways to facilitate ion migration within the electrolyte. The morphology with 90 mol % LiBF₄ (FIG. 16c) is significantly different, with more grains and grain boundaries that illustrate the plasticity of this solid-state electrolyte. FIGS. 16d and 16e present the static solid state ⁷Li NMR spectra of the 10 and 90 mol % LiBF₄ in ZIPC1, measured over a temperature range of 20 to 60° C. which is below the melting point. Commonly, solid samples give ⁷Li spectra with broad line shapes due to strong homonuclear Li—Li interactions. However, the existence of mobile components, due to the increased disorder or the presence of an amorphous phase, leads to narrowing of the line shapes. The single pulse ⁷Li spectra of 10 mol % LiBF₄ in ZIPC1 show a narrow line shape that suggests the dipolar interactions are well averaged. The ⁷Li spectra of 90 mol % LiBF₄ in ZIPC1, however, shows a narrow component superimposed on top of a broad component, attributed to the presence of both mobile and less mobile Li ions, respectively. FIGS. 16f and 16g present the single pulse static ¹⁹F NMR spectra of the 10 and 90 mol % LiBF₄ in ZIPC1. The ¹⁹F peak from ZIPC1 gives a broad line that overlaps with the line shape from the BF₄ due to their close chemical shifts. In both samples, the width of the broader component (which is relatively narrow compared to that expected for fully ordered materials), and the substantial amount of narrow component, indicate substantial mobility/disorder of both the BF₄ anions and the —BF₃ groups on ZIPC1. The ionic conductivity of the mixtures with LiBF₄ is around 3 orders of magnitude higher than for the pure ZIPC1, due to the presence of a higher concentration of charge carriers in the electrolyte (FIG. 17a). The self-diffusion coefficient of ⁷Li and ¹⁹F were measured using pulse-field gradient (PFG) NMR at different temperatures, presented in FIG. 17b. In the 10 mol % LiBF₄ in ZIPC1, the diffusion of BF₄ anions is faster than the diffusion of the ⁷Li cations. By increasing the salt concentration to 90 mol %, the ⁷Li cations become the fastest diffusing entity. This is predicted to be beneficial for performance of the electrolyte in lithium batteries. Cyclic voltammetry (CV) was used to investigate the compatibility and electrochemical stability of these new electrolytes with Li metal. Both 10 and 90 mol % LiBF₄ in ZIPC1 electrolytes showed stable cycling behaviour, plus clear Li deposition and stripping peaks with small peak separation. This demonstrates that this new class of electrolytes can support the fast, stable and reversible reduction and oxidation reaction of Li/Li⁺ without substantial parasitic side reactions. The peak currents for the 10 mol % LiBF₄ sample is lower than for 90 mol %, as expected from the lower ionic concentration in the electrolyte (FIG. 18). Thermal studies—The thermal properties of the neat ZIPCs, various lithium doped composites and protic ZIPC are given in Table 1. Table 1 describes the thermal properties of several ZIPCs whereby the presence of a solid-solid phase transition (at Tₛ₋ₛ) is one piece of evidence of potential structural disorder. Notably, alkylation of the ring results in an advantageous reduction in Tₛ₋ₛ compared to the protonated salts, while the Tₘ remains above room temperature for both ZIPCs after doping, which is important for their use as solid-state electrolytes.

TABLE 1
Thermal Properties of ZIPCs

| Cpd | | | |
|---|---|---|---|
| # | Zwitterion structure | Ts-s/° C. | Tₘ/° C. |
| 7 | 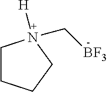 | 91 | 107 |
| 1 | 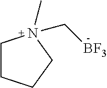 | 54 | 98 |
| 1 | 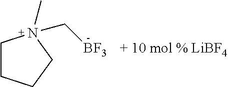 | 48 | 62 |
| 1 | 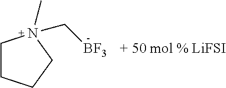 | T_{g} = 59 | — |
| 2 | 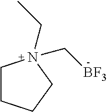 | 48 | 65 |
| 2 | 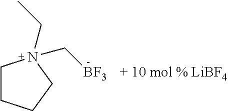 | T_{g} = −63, Tₓ ≈ 5 | 38 |
| 1 | 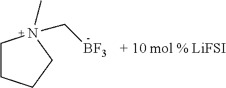 | T_{g} = −66, Tₛ₋ₛ ≈ 20 | 67 |
| 1 | 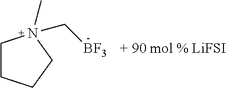 | T_{g} = −66, Tₛ₋ₛ ≈ 32 | 77 |
| 1 | 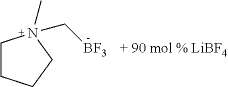 | T_{g} = −27, Tₛ₋ₛ = 27, Tₛ₋ₛ = 132 | 220 |
| 6 | 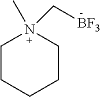 | 105 | >145 |
| 5 | 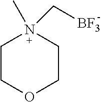 | Tₛ₋ₛ = 25 | 120 |
| 5 | 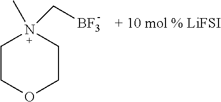 | T_{g} = −29, Tₛ₋ₛ = 25 | 92 |

The ZIPCs with the BF₃ group demonstrate the presence of a solid-solid phase transition in both compounds 1 and 2 (FIG. 2, Table 1). This behaviour is one key indicator of plasticity (when observed with at least one other characteristic indication, e.g., from NMR or SEM) as these transitions represent the onset of a disordering mechanism (e.g. rotation of specific functional groups) that goes hand-in-hand with the formation of vacancies in the material and increased conductivity.

The DSC trace of ZIPC1 (C₂mpyrBF₃) in FIG. 1Ba shows the onset temperatures and entropy changes for each transition. As can be seem, the thermal analysis shows one distinct solid-solid phase transition peak which differentiates two solid phases before the melt at 98° C. The solid-solid phase transition for ZIPC1 shows relatively low entropy change of 13 J mol ⁻¹K⁻¹. The entropy change of the melting transition is 21.4 J K⁻¹ mol⁻¹, which is close to the 20 J mol ⁻¹K⁻¹ required by Timmerman's criterion for plastic crystalline behaviour indicating significant disorder in the material in phase I (the highest temperature solid phase before melt).

The DSC trace of ZIPC2, the onset temperatures and entropy changes for each transition are shown in FIG. 1Bb. As can be seen ZIPC2 also shows a solid-solid phase transition, this time at 45° C. The existence of this phase transition represents the onset of molecular rotations within the material, through which the material can be disordered. An increase in the length of alkyl chain substituent produces a decrease in melting point from 98° C. in ZIPC1 (C₂mpyrBF3) to 60° C. in ZIPC2 (C₂epyrBF3).

For ZIPC6, the DSC trace of ZIPC6, the onset temperatures and entropy changes for each transition are shown in FIG. 1Bd. ZIPC 6 displays a peak at 105° C. in the DSC trace. Monitoring this sample visually at temperatures above 100° C. (as can be seen in the photos in FIG. 1B) revealed that the peak at 105° C. is not a melting transition as the sample is solid even at 145° C. It is a solid-solid transition.

Each of ZIPC1, ZIPC2, ZIPC5, and ZIPC6 display a solid-solid phase transition before melt. The existence of this transition, along with the low entropy of melting of ZIPC1, are well-known characteristics of plastic crystal behaviour. Typically, well-ordered crystalline organic salts do not have solid-solid phase transitions in the solid phase and have ΔSₘ>60 J mol⁻¹ K⁻¹.¹

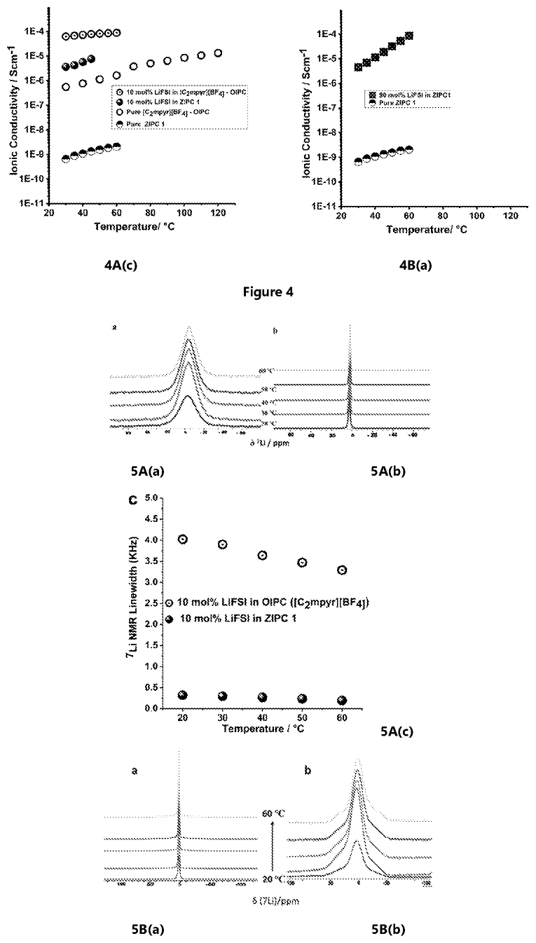

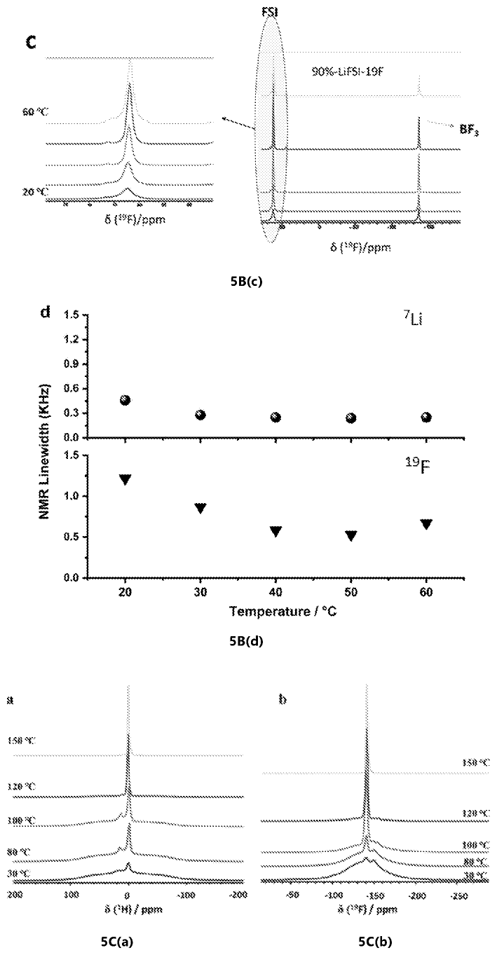

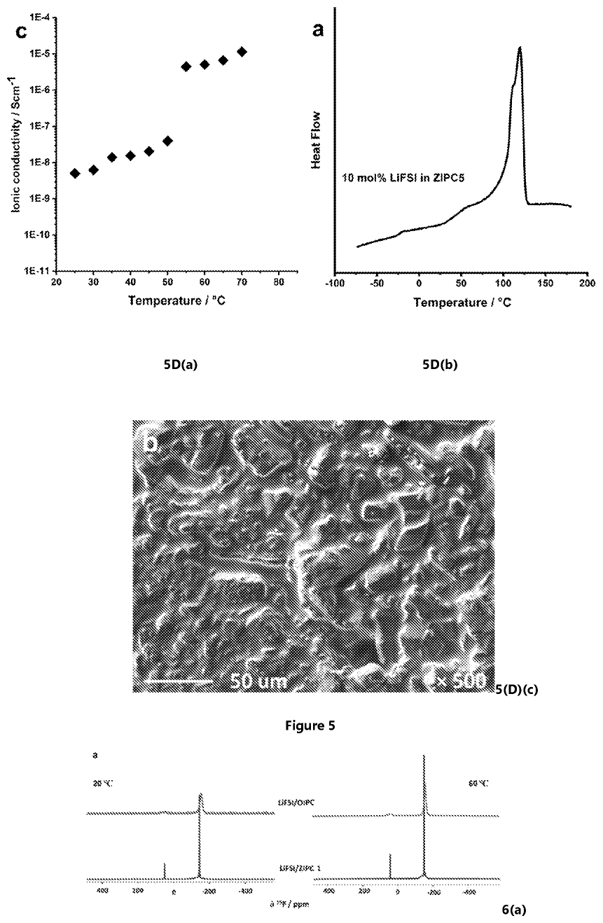

Example 1—Thermal phase behaviour—10 mol % LiFSI doped ZIPC1 electrolyte—The thermal phase behaviour of ZIPC1 and 10 mol % LiFSI doped ZIPC1 is compared with that of pure [C₂mpyr][BF₄] OIPC and 10 mol % LiFSI doped [C₂mpyr][BF₄]OIPC in FIG. 2A. ZIPC1 shows a solid-solid phase transition (at 54° C.) which is one important characteristic of plastic crystal behaviour. The ZIPC has an onset of melt at 98° C., with an entropy of fusion, ASf, of 21.4 J K-1 mol-1. The ASf value is very close to Timmermans criteria of plastic crystal behaviour, and less than that of many known 01PCs. The analogous OIPC [C₂mpyr][BF4] decomposes at 250° C. before melting. Thus, the thermal analysis (and NMR data described later) supports the assignment of this new zwitterion structure as a plastic crystal. Doping ZIPC1 with 10 mol % LiFSI decreases Tₘ, to 59° C. with a small ΔS_{f} of 10 J K⁻¹ mol⁻¹. A glass transition (Tg) was also observed at −66° C. indicating the appearance of an amorphous phase in the mixture. The addition of 10 mol % LiFSI to [C₂mpyr][BF₄] OIPC introduces additional new peaks at low temperatures (−95° C. and −70° C.) below the phase IV to III transition and also at 83° C. after the II-I transition suggesting of formation new phases at low temperature (FIG. 2A(b)). The formation of new phases after lithium salt addition has previously been observed in other Li mixtures of pyrrolidinium based OIPCs and is not unique to ZIPCs. Initial indications are that this material formed by combination of the OIPC and Li salt is a new, homogeneous solid rather than a solid/liquid combination as is formed with the ZIPC and salt combination. As a result, slower Li⁺ transport is expected in the OIPC electrolyte (as it is moving through a solid rather than a liquid or amorphous phase) which is supported by the wider linewidths seen in the NMR spectra (FIGS. 4 and 5), discussed below.
Example 2—Thermal phase behaviour—90 mol % LiFSI and ZIPC electrolyte mixture—DSC heating traces of ZIPC1 and an electrolyte mixture of 90 mol % LiFSI in ZIPC1 are shown in FIG. 2B. Adding only 10 mol % ZIPC1 to LiFSI decreases Tₘ to 77° C. Furthermore, a glass transition (Tg) was also observed at -66 ° C. indicating the appearance of an amorphous phase.
SEM Analysis—ZIPC1—The SEM images of ZIPC1 show slip steps and/or glide planes that normally can be seen in the OIPCs due to their plastic nature which cannot be seen in a normal organic/inorganic crystals like sodium fluorite that are hard and brittle (FIG. 3D). Since the SEM image was taken at room temperature, the material is expected to have increased plasticity at higher temperatures. ¹⁹F NMR spectra shows strong evidence of higher level of plasticity at higher temperatures as the linewidth get narrow gradually as well as a fraction of narrow component appeared at 40° C. and grew in proportion with increasing temperature. All these results show that ZIPC1 has an inherent rotational motion of molecules and make a disorder phase in ZIPC.

Furthermore, the microstructure/morphology of the surface of ZIPC1 pellet shows evidence of plasticity as several grains with different orientations can be observed. Furthermore, some sets of slip planes within different grains can be seen. These slip steps are also observed in plastic OIPC systems. The grain boundaries are clearly detected from the fractured surface of ZIPC6. The slip steps retained their coherency until terminate grain boundaries also contributes plasticity. Given that the SEM image obtained at room temperature, which is phase II for the two ZIPCs (not the highest temperature solid phase), this suggests that there will be a higher level of plasticity at higher temperature. This is consistent with the slight increase in the mobile component observed in the ¹⁹F NMR measurements with increasing temperatures, discussed below.

SEM Analysis—Example 1-10 mol % LiFSI doped ZIPC1 electrolyte—SEM analysis of pure ZIPC1 (10 mol % LiFSI in ZIPC1 are shown in FIG. 3A. The microstructure of the surface of the ZIPC1 pellet shows evidence of plasticity as several grains with different orientations can be observed (FIG. 3a). Furthermore, some sets of slip planes within different grains can be seen. These slip steps have also been observed in plastic OIPC systems. SEM images of 10 mol % LiFSI doped ZIPC1 also suggest that the particles in the ZIPC1 electrolyte are connected by a new, liquid-like phase (FIG. 3b). Based on the NMR data (below) it is proposed that this phase has high concentration of LiFSI. Thus, this phase is believed to provide pathways for Li ion diffusion and facilitates target ion transport through the electrolyte. SEM images of this mixture also suggest this, and that the particles in the ZIPC1 electrolyte are connected by this new, liquid-like phase. Based on the NMR data (below) it is believed that this phase has a high concentration of LiFSI. Thus, this phase provides pathways for Li ion diffusion and facilitates target ion transport through the electrolyte.
SEM Analysis—Example 2—90 mol % LiFSI and ZIPC electrolyte mixture—SEM images of an electrolyte mixture of 90 mol % LiFSI in ZIPC1 are shown in FIG. 3B. The SEM images of the electrolyte mixture of 90 mol % LiFSI in ZIPC1 show crystalline regions and intergranular regions contain mobile, Li rich electrolyte providing pathway for lithium ions which supports the lithium electrochemistry and device cycling.
Example 3—ZIPC5 and 10 mol % LiFSI in ZIPC5—The static ¹H and ¹⁹F NMR spectra of pure ZIPC5 (methylated morpholinium compound) shows evidence of disorder as a narrow linewidth exists even at 30° C., the level of disorder is higher at higher temperatures, demonstrated by the narrower linewidths and increased fraction of narrow component. This disorder (FIG. 5D(a)-(c) is consistent with the material being a plastic crystal. The DSC trace of neat ZIPC5 shows a broad peak around 25° C. that can be attributed to a solid-solid phase transition and a sharp melting peak at 120 ° C. The SEM image of ZIPC5 shows grain boundaries that can be an evidence of plasticity as these grain boundaries cannot be seen in fully ordered, crystalline materials. The existence of the grain boundaries in the structure of ZIPCs can assist ion conduction. All these results show that ZIPC5 has a disordered structure consistent with it being a plastic crystal.

The solid-solid phase transition around 25° C. is more prominent in the 10 mol % LiFSI in ZIPC5 sample. Adding only 10 mol % LiFSI into ZIPC5 decreases the melting point to 92° C. Furthermore, a glass transition (Tg) was also observed at −29° C. indicating the appearance of an amorphous phase. SEM image of 10 mol % LiFSI in ZIPC5 shows a new amorphous phase that could provide pathways for Li ion diffusion and facilitate target ion transport through the electrolyte and would be very beneficial for the application of the material as an electrolyte in Li batteries. The ionic conductivity of 10 mol % LiFSI in ZIPC5 shows that there is a jump in conductivity at 50° C., suggesting higher mobility of Li and FSI ions after the solid-solid phase transition of ZIPC5—similar behaviour has previous been observed in other plastic crystal materials. As the ionic conductivity of the neat ZIPC5 is not measurable, this ionic conductivity can be attributed to the mobility of the FSI anions and Li cations.

Transport and electrochemical properties of ZIPC1 and as a quasi-solid-state electrolyte—Evaluation of the electrochemical properties and interfacial behaviour of the novel electrolytes containing ZIPCs was based on: (i) voltammetric characterisation of the behaviour of three-electrode cells, with Li metal as the working electrode, (ii) galvanostatic and EIS characterization of symmetrical Li metal coin cells to demonstrate the applicability of these unique electrolyte materials. Measurement of the Li⁺ transference number was performed electrochemically by chronoamperometry techniques and compared to the NMR results when applicable. Comparison of t_{Li+} between the ZIPCs and OIPCs provide important preliminary confirmation of the benefits of zwitterions for improved target ion transport.
Ionic conductivity and NMR linewidths—Example 1-10 mol % LiFSI doped ZIPC1 electrolyte - The ionic conductivity of the 10 mol % LiFSI doped [C₂mpyr][BF₄] electrolyte is approximately one order of magnitude higher than ZIPC1/LiFSI mixture (FIG. 4A). This higher conductivity was expected as the OIPC-based electrolyte is composed entirely of individual ions, whereas in the ZIPC1 electrolyte, 90% of the ionic component are tethered and thus cannot migrate in an electric field such that only Li and FSI ions can move. Indeed, the fact that the conductivity of the ZIPC-based electrolyte is so close to that of the OIPC is quite remarkable and points to significant mobility of the Li cations and FSI anions in the doped ZIPC matrix material. This was further analysed by NMR, as discussed below.

Measurement of the linewidths of static NMR spectra indicates the relative mobility of the NMR active nuclei. Thus, although OIPCs are solid materials, their intrinsic disorder (e.g. significant rotational motion of the cations and/or anions) results in significantly narrow lines typically of those observed in crystalline solids. It should be noted that a fully liquid sample give very narrow lines as all species are fully mobile, with both translational and rotational motion. Notably, the static NMR linewidths for Li are much broader in the 10 mol % LiFSI doped [C2mpyr][BF4] OIPC electrolyte than they are in the equivalent 10 mol % LiFSI doped ZIPC1 electrolyte. While overall, the OIPC-based electrolyte is more ionically conductive because it contains more free ions, the doped lithium ions are much less mobile than they are in the salt doped ZIPC1 electrolyte.

In contrast, the ⁷Li spectra for the Li FSI doped OIPC electrolyte exhibit a relatively broad single peak at 20° C., and a very small fraction of second, narrow component appears at 30° C. and increased very slightly at 60° C. (FIG. 5A(a)). This suggests that there is very small proportion of diffusive Li ions (although not enough to measure ⁷Li diffusion coefficient). In contrast, the ⁷Li spectrum for LiFSI doped ZIPC electrolyte shows only a narrow signal for whole range of temperatures (linewidth of around 0.3 KHz or less) and remains reasonably constant with increasing temperature (FIG. 5A(b)). This indicates that the majority of Li ions in the LiFSI/ZIPC1 mixture are quite mobile, which is consistent with the hypothesis of a lithium-rich liquid-like phase in the LiFSI/ZIPC electrolyte. The linewidths for Li are much broader in the 10 mol % Li FSI doped OIPC electrolyte than they are in the equivalent doped ZIPC1-based electrolyte (FIG. 5A(c)). Thus, although overall the OIPC-based electrolyte is more conductive, because it contains more free ions, the lithium ions appear to have much less mobility than they do in the ZIPC1-based electrolyte.

This is also supported by the ¹⁹F NMR, shown in FIG. 5. In brief, the ¹⁹F spectra of the LiFSI doped OIPC electrolyte at 20° C. exhibits one broad peak for BF₄ and one very small broad peak for FSI ion. However, at 60° C., the spectra indicate two different BF₄ environments, representing a relatively mobile component and a less mobile component. In contrast, the ¹⁹F spectra of the BF₃ group in the LiFSI doped ZIPC1 electrolyte indicate the presence of both a mobile and less mobile component at all temperatures studied. Further, the ¹⁹F spectra of the FSI anion in LiFSI doped ZIPC1 electrolyte has only one narrow peak (i.e. representing one mobile component) over the whole temperature range examined. This suggests that almost all FSI anions are diffusive, which is supported by the measured diffusion coefficient of FSI that increases from 3×10⁻¹³ to 4.6×10⁻¹² m²s⁻¹ over the temperature range studied.

The ¹H spectra for the 10% LiFSI doped ZIPC1 electrolyte (not shown) also supports the hypothesis of two phases. However, the spectra are dominated by narrow sharp line at all temperatures suggesting the majority of cations are mobile, most probably in the liquid phase. In contrast, although the ¹H spectrum for the LiFSI doped OIPC electrolyte mixture also indicates the presence of cations with significant mobility, these are present at very low concentrations e.g. only 2% narrow component at 40° C., compared to 60% in the LiFSI doped ZIPC1 electrolyte.

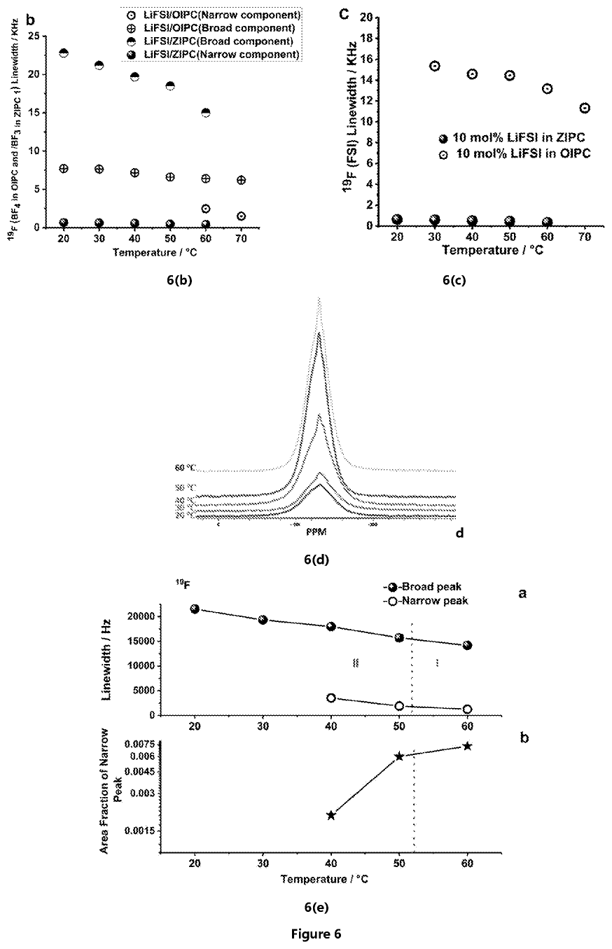

In FIG. 6a, it can be seen that the peaks gradually narrow (smaller linewidth) with increasing temperature from 21.5 KHz at 20° C. to 14.1 KHz at 60° C. This indicates more “mobility” (attributed to rotational disorder) of the —BF₃ species as the material is warmed up. A second narrow peak (with a line width of ca. 1.2 KHz) on top of the initial broad peak becomes distinguishable from 40° C., suggesting that there is a small but growing proportion of dynamic anions present in the higher temperatures (in phase I) (FIG. 6d-e). The presence of broad and narrow components is not unique to ZIPCs, but is a clear indicator of disorder within the plastic crystal.

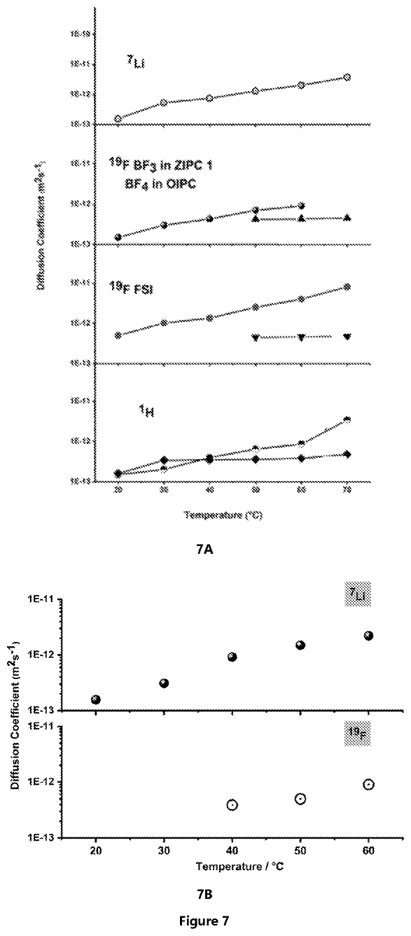

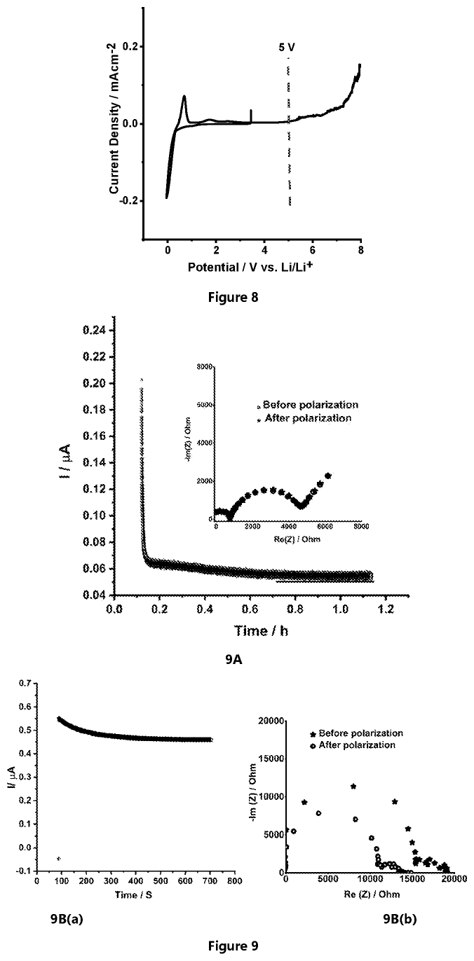

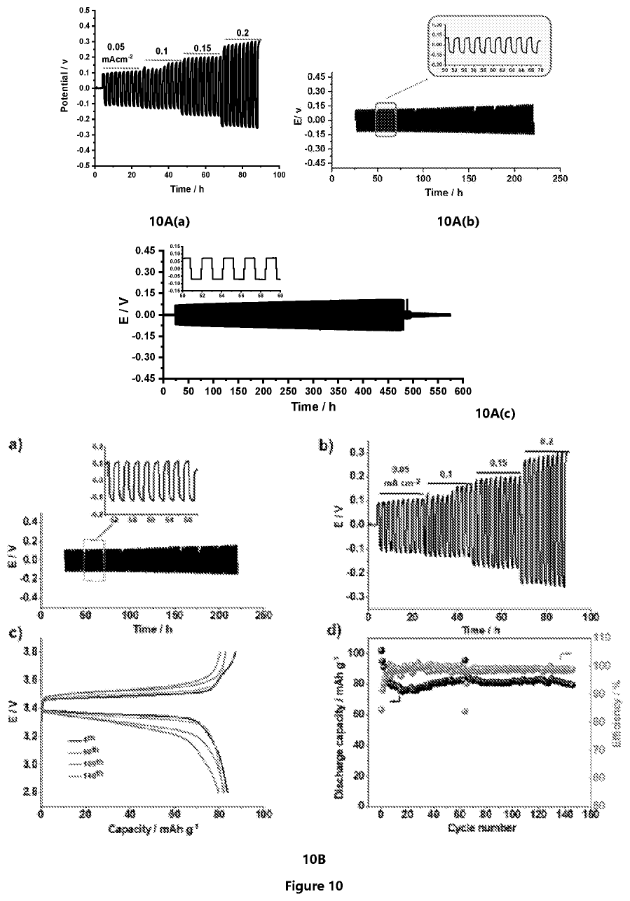

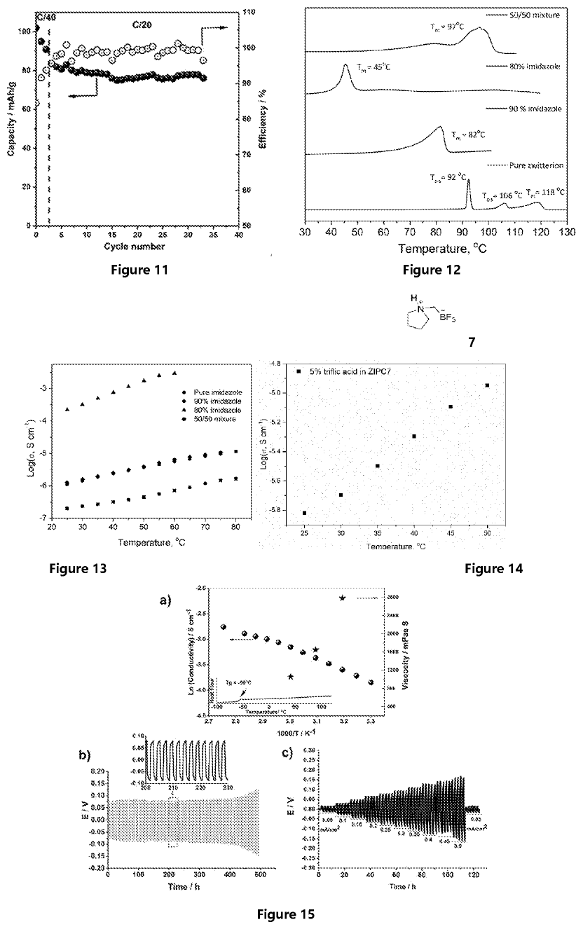

Ionic conductivity and NMR linewidths—Example 2—90 mol % LiFSI and ZIPC electrolyte mixture—The ionic conductivity of pure ZIPC1 and an electrolyte mixture of 90 mol % LiFSI in ZIPC1 as a function of temperature is shown in FIG. 4B. As both pure LiFSI and pure ZIPC have very low ionic conductivity, this result shows that by addition of only 10 mol % ZIPC1 the ionic conductivity of this mixture increased significantly. The ⁷Li spectra of a 90 mol % LiFSI and ZIPC1 electrolyte mixture, ⁷Li spectra of pure LiFSI and the ¹⁹F spectra of 90 mol % LiFSI and ZIPC1 electrolyte mixture, and ⁷Li and ¹⁹F linewidth of 90 mol % LiFSI and ZIPC1 electrolyte mixture as a function of temperature are shown in FIG. 5B. The ⁷Li spectrum for the 90 mol % LiFSI and ZIPC1 mixture shows only a narrow signal for whole range of temperatures (around 0.3 KHz or less) and remains reasonably constant with increasing temperature (FIG. 5B(a)). This indicates that the majority of Li ions are quite mobile in this electrolyte. In contrast, the ⁷Li spectra for pure LiFSI exhibits a broad single peak at the whole range of temperatures, indicating quite low mobility (FIG. 5B(b)). The ¹⁹F spectra of FSI anion in 90 mol % LiFSI and ZIPC1 electrolyte mixture has only one narrow peak (i.e. a mobile component) over the whole temperature range (FIG. 5B(c)). This suggests that almost all FSI anions are diffusive, supported by the measured diffusion coefficient of FSI. The linewidths of both ⁷Li and ¹⁹F are small in the 90 mol % LiFSI and ZIPC1 electrolyte mixture, indicating quite high mobility (FIG. 5B(d)).
Diffusion coefficients—Example 1—10 mol % LiBF₄ doped ZIPC1 electrolyte—The diffusion coefficients show that the Li and FSI diffuse faster in the LiFSI doped ZIPC1 electrolyte than in the LiFSI doped OIPC electrolyte (FIG. 7A). This is consistent with those anions being predominantly in a liquid phase in the former. It is also important to note that only a small fraction of ions were sufficiently mobile in the LiFSI doped OIPC electrolyte to be measurable, the ¹⁹F NMR could only be measured above 50° C. The diffusion coefficients show that the Li diffusion is not measurable in the doped OIPC-based electrolyte even at high 60° C. Diffusion of FSI ions can only be measured above 50° C., which is consistent with NMR linewidth that shows only a small fraction of FSI ions above 50° C. were sufficiently mobile in the doped OIPC electrolyte to diffuse. The significantly higher diffusion rates in the LiFSI doped ZIPC1 electrolyte clear indication the utility of ZIPCs for lithium battery applications, which is explored further below.
Diffusion coefficients—Example 2—90 mol % LiFSI and ZIPC1 electrolyte mixture—FIG. 7B illustrate ⁷Li and ¹⁹F diffusion coefficients of a 90 mol % LiFSI and ZIPC1 electrolyte mixture at different temperatures measured by PFG-NMR. The diffusion coefficients show that the ⁷Li diffuse faster than ¹⁹F in the 90 mol % LiFSI and ZIPC1 electrolyte mixture. This indicates the Li transference number is high in this electrolyte.
Electrochemical studies—Example 1—10 mol % LiFSI doped ZIPC1 electrolyte—Cyclic voltammetry (CV) was used to investigate the Li plating (negative scan) and stripping (positive scan) behaviour using 10 mol % LiFSI doped ZIPC1 electrolyte. The CV data (FIG. 8) shows successful stripping and plating of Li metal, and that this is stable and reversible and the current density remains stable during successive cycling. Electrochemical stability of electrolyte is an important factor for the electrochemical devices. The results show that 10 mol % LiFSI doped ZIPC1 exhibits an anodic limit of 5 V (vs. Li/Li+). It shows that this electrolyte has wide enough electrochemical window to be used in cells with high voltage cathode materials. In addition, this demonstrates the ability of this electrolyte to support reversible striping and plating of the Li/Li+ couple. The electrochemistry is examined further below. Chronoamperometry of the Li|10 mol % LiFSI doped ZIPC1 electrolyte|Li cells at a potential step of 10 mV at 50° C. (FIG. 9A). The lithium transference number (t_{Li}+) value was found to be 0.3. These are significant transference numbers for Li⁺. It should be noted that t_{Li}+ is not possible to measure in 10 mol % LiFSI doped OIPC.
Electrochemical studies—Example 2—90 mol % LiFSI and ZIPC1 electrolyte mixture—Chronoamperometry studies of Li 190 mol % Li FSI in ZIPC1 electrolyte mixture 1 Li cells at a potential step of 10 mV at 50° C. are shown in FIG. 9B. The inset is Nyquist Profiles of the cell's electrochemical impedance spectroscopy response before polarization and after the steady-state current. The lithium transference number (t_{Li}+) value was found to be 0.7. This is a significantly high transference number for Li⁺ and demonstrates the promise of the zwitterionic plastic crystal compounds for electrolyte formation.
Cycling studies—Example 1—10 mol % LiFSI doped ZIPC1 electrolyte—The 10 mol % Li FSI doped ZIPC1 electrolyte was then trialled in a symmetrical lithium metal cell (FIG. 10A(a)). The polarization of the cell increases with increasing current density, as is normal behaviour. The voltage profiles, however, are symmetric and reversible at all current densities. Thus, the results indicate that electrolyte is highly compatible with reactive lithium electrodes and is able to support Li ions transport for even 0.2 mAh cm⁻² charges. FIG. 10 shows that the electrolyte cycled well even at higher applied current density (0.2 mAcm⁻²). These results show that this electrolyte is a good candidate to act as an electrolyte for lithium batteries, supporting the high voltage electrochemistry of lithium as well as providing facile lithium ion transport; and b) symmetric cell cycling performance of 10 mol % LiFSI doped in ZIPC1 at 0.1 mA/cm² at 50° C. The charge-discharge interval was kept at 1 hr. The inset is a zoom of voltage profile at cycles 50-70. Thus, this electrolyte demonstrates stable cycling with low polarisation potential for 100 cycles. The electrolyte also exhibited good stability and reversibility even at 0.1 mA cm⁻² applied current density for 100 cycles (FIG. 10A(b)) demonstrating excellent cell performance. A full cell consisting of a lithium metal anode with a lithium iron phosphate (LFP) cathode, LFP|10 mol % LiFSI in ZIPC|1 Li was cycled at 50° C. in the range of 2.8 to 3.8 V (FIG. 11). This cell exhibited stable long-term cycling at C/20 at 50° C. The cell shows an increase in reversible capacity with cycling. It delivers reversible discharge capacity of 5 mAh/g in the first cycle and it reaches a reversible discharge capacity of 24 mAh/g in 70th cycle. It is postulated that an increase in reversible capacity might be a result of internal heating during cycling, causing melting of the electrolyte near the interface of electrode leading to better wetting of the electrode material with electrolyte. Capacity retention of 90% with 98% columbic efficiency was achieved over 30 cycles. These results show promising preliminary charge-discharge cycling performance for LFP|10 mol % LiFSI doped ZIPC1 electrolyte|Li cell at 50° C. The unoptimised cell shows remarkable efficiency (average efficiency is 98%) which is very important for battery performance.
Cycling studies—Example 2—90 mol % LiFSI and ZIPC electrolyte mixture—FIG. 10B illustrates symmetric cell cycling performance of a 90 mol % LiFSI and ZIPC1 electrolyte mixture at 0.1 mA/cm2 @50° C. The charge-discharge interval was kept at 1 hr. The inset to FIG. 10(A)(c) is a zoom of the voltage profile at cycles 50-60. This electrolyte demonstrates stable cycling with low polarisation potential for 480 cycles. FIG. 12 illustrates that a DSC trace of ZIPC7 shows 3 peaks in the heating cycle (T1=92° C.; ΔHf=26 J/g; T2=106° C.; ΔHf=10 J/g; T3=119° C.; ΔHf=25 J/g)(Melting point of imidazole=89° C.);) DSC of 50/50 mixture of ZIPC7/imidazole shows one broad melting peak at 97° C. with accompanying ΔH_{f}=25 J/g. This is different from the trace of the pure ZIPC7, and no peak for pure imidazole is present (Tₘ=89° C.). Thus, the changed melting behaviour confirm interactions occurring between imidazole and zwitterion.
FIG. 13 illustrates a) Conductivity of pure protic zwitterion ZIPC7, and upon doping with imidazole base. The conductivity of each sample was measured in triplicate. Pure imidazole shows the lowest conductivity among all samples. In all cases the conductivity increases with temperature. Small addition of zwitterion (10%) to imidazole give 10 times higher conductivity. The highest conductivity was obtained when 20% of zwitterion was added to imidazole. In this case, at room temperature, conductivity was about 1000 times more than conductivity of pure imidazole. The conductivity of 90/10 mixture is similar to 50/50 mixture.
Protic ZIPC electrolytes -To investigate the benefits of protic ZIPCs as anhydrous proton conductors, ZIPCs have been doped with either acid or base. For example, ZIPC7 was doped with triflic acid. The conductivity after triflic acid doping was determined to be 10⁻⁶ to 10⁻⁵ S cm⁻¹. The CV showed some electrochemical (H) activity which is very important for fuel cell applications. However, doping with the solid base imidazole appears to be more promising (see FIG. 13) and this is summarised below. DSC of 50/50 mixture (see FIG. 12) shows one broad melting peak at 97° C. with accompanying ΔHf=25 J/g. The sample looks different than pure zwitterion, with only one peak present. Pure imidazole peak also not present (Tm=89° C.). Changed melting behaviour confirm interactions occurring between imidazole and zwitterion.
Comparison of conductivity with different combinations—Pure imidazole shows the lowest conductivity among all samples (bottom set of circles in FIG. 13). In all cases conductivity increases with temperature. Small addition of zwitterion ZIPC7 (10%) to imidazole increases the conductivity 10 times (from 2.01×10⁻⁷ S/cm to 10⁻⁶S/cm, middle set of circles in FIG. 13). The highest conductivity was obtained when 20% of zwitterion ZIPC7 was added to imidazole (top set of triangles in FIG. 13). In this case, already at room temperature, conductivity was 2.23×10⁻⁴ S/cm, which is about 1000 times more than conductivity of pure imidazole. The conductivity of 90/10 (middle set of circles in FIG. 13) mixture is similar to 50/50 mixture (set of diamonds behind I:ZI 90:10 circles).

Thus, in conclusion, the base-doped ZIPC7 shows much higher conductivity than neat imidazole (the pure ZI is too low to be measurable). These conductivities are good for a solid state, anhydrous proton conductor. As pure imidazole is often used for proton conduction, this means that this protic ZIPC may provide significant improvement in terms of proton conduction than pure imidazole.

Zwitterion-based liquid electrolytes—To explore the efficacy of using zwitterions as non-volatile media for high target ion conduction in a liquid electrolyte, a high lithium salt content was used in combination with pyrrolidinium ZIPC1. With 50 mol % LiFSI in ZIPC1, only a T_{g} at −59° C. is present (inset FIG. 15a) and the material is liquid at room temperature. Thus, this zwitterion forms a high salt content liquid electrolyte. The zwitterion-based electrolyte is non-volatile and has no competing cation migration. The prior work on the development of zwitterionic liquids as an electrolyte medium has predominantly used sulfonate or sulfonyl imide anions in combination with imidazolium cations, with the best results achieved using a linker of between five to seven CH₂ groups. It was considered that the smaller size of the ZIPC1 molecule and use of the charge-diffuse BF₃ moiety would enhance the conductivity and transference number. Indeed, the conductivity of the new material is 1.4×10⁻⁴ S cm⁻¹ (30° C.), FIG. 15a, which is the same as or higher than other liquid non-plastic zwitterion electrolytes reported, and it also has a high transference number of 0.55±0.05 at 50 ° C. The new zwitterion liquid electrolyte also supports excellent stability for cycling lithium metal (FIG. 15b) and is believed to be the first proof of lithium metal cycling for a liquid zwitterion electrolyte. A range of current densities, up to 0.5 mA cm⁻², were applied for one hour for 5 cycles at each current. The stripping and plating of lithium occurs with good stability and low polarisation potential even at 0.5 mA cm⁻². Importantly, when the current density was returned to 0.05 mA cm⁻², the low overpotential was recovered. This stability was also retained with longer term cycling at 0.2 mA cm⁻² (0.2 mA h cm⁻²). The overpotential remained low and stable at ˜80 mV, even decreasing to ˜70 mV after 65 cycles. This is attributed to the low internal resistance and is consistent with the formation of a conductive SEI layer.

### Synthesis of ZIPC3

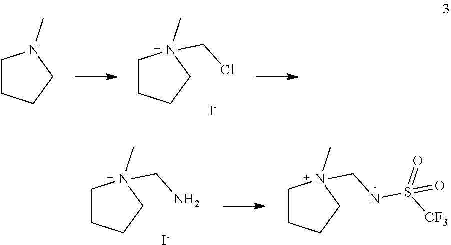

1-(chloromethyl)-1-methylpyrrolidin-1-iumiodide-1-methylpyrrolidine (1 eq.) in ethyl acetate was reacted with chloroiodomethane (1 eq.) and stirred at room temperature under an inert atmosphere for 16 h. The ethyl acetate was then removed in vacuo, the solid washed with diethyl ether to give the product as a light-brown solid (98% yield). ¹H NMR (run Jul. 2 2018) (400 MHz, CDCl₃): 5.80; (s, 2H, NCH₂Cl), 4.15-4.21; (m, 2H, CH₂-5(Pyr)), 3.88-3.93; (m, 2H, CH₂-2 (Pyr)), 3.50; (s, 3H, NCH₃), 2.32-2.45; (m, 4H, CH₂-3,4 (Pyr)). ¹³C NMR (run Dec. 3 2018) (100.6 MHz, CDCl₃): 68.98, 64.35, 49.18, 22.37
1-(aminomethyl)-1-methylpyrrolidin-1-ium iodide-1-(chloromethyl)-1-methylpyrrolidin-1-ium iodide was reacted with a solution of aqueous ammonia (28%) and stirred at room temperature for 36 h. The solvent was removed under vacuum, the resulting residue washed with dichloromethane and dried in vacuo to obtain the target structure as a brown resin. (˜30% yield). ¹H NMR (run Feb. 9 2019) (400 MHz, CDCl₃): 5.61; (s, 2H, NCH₂NH₂), 4.17-4.19; (m, 2H, CH₂-5 (Pyr)), 3.84-3.88 3.88-3.93; (m, 2H, CH₂-2 (Pyr)), 3.46; (s, 3H, NCH₃), 2.37-2.46; (m, 4H, CH₂-3,4 (Pyr))
((1-methylpyrrolidin-1-ium-1-yl)methyl)((trifluoromethyl)sulfonyl)amide-1-(aminomethyl)-1-methylpyrrolidin-1-ium iodide (1 eq.) in dry acetonitrile was reacted with a solution of trifluoromethylsulfonyl chloride (1.5 eq.) in acetonitrile at ˜0° C. . The mixture was then stirred for 4 days under an inert atmosphere at RT before drying under vacuum. The resulting residue was purified with dichloromethane/water before drying under vacuum to obtain the target structure as a brownish resin. ¹H NMR (19 Dec. 19) (400 MHz, CDCl₃): 5.44; (s, 2H, NCH₂NHS), 3.95-3.99; (m, 2H, CH₂-5 (Pyr)), 3.74-3.79; (m, 2H, CH₂-2 (Pyr)), 3.37; (s, 3H, NCH₃), 2.34-2.39; (m, 4H, CH₂-3,4 (Pyr)). ¹⁹F NMR (19/12/19) (376.5 MHz, CDCl₃): −78.60

### Synthesis of ZIPC4

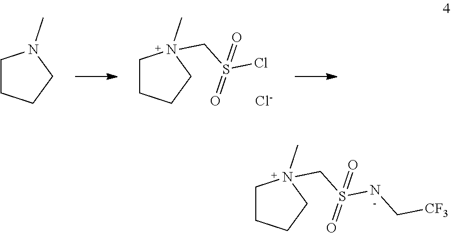

1-((chlorosulfonyl)methyl)-1-methylpyrrolidin-1-ium chloride N-methylpyrrolidine (1 eq.) in dry dimethylformamide was reacted with cold chloromethane sulfonylchloride (1.2 eq.) then the solution stirred for 3 days under an inert atmosphere at RT. The product was then dried in vacuo, washed with diethyl ether to obtain a black tar. The tar was then taken immediately on to the next step due to sensitivity of the sulfonyl chloride moiety.
(((1-methylpyrrolidin-1-ium-1-yl)methyl)sulfonyl)(2,2,2-trifluoroethyl)amide-1-((chlorosulfonyl)methyl)-1-methylpyrrolidin-1-ium chloride (1 eq.) in anhydrous dichloromethane was reacted with 1,1,1-trifluoroethylamine (1.2 eq.) in a suspension of sodium bicarbonate (1.8 eq.) and anhydrous dichloromethane. The reaction was stirred under an inert atmosphere at room temperature for 48 h before the solids were filtered off and the filtrate organics removed under vacuum. The resulting residue was washed 3 times with diethyl ether and dried under vacuum to obtain the target structure, as a brown solid (˜50% yield). ¹H NMR (Jun. 8 2018) (400 MHz, CDCl₃): 5.61; (s, 2H, NCH₂SO₂), 4.34; (s, 2H, NCH₂CF₃), 4.01-4.07; (m, 2H, CH₂-5 (Pyr)), 3.77-3.83 3.74-3.79; (m, 2H, CH₂-2 (Pyr)), 3.39; (s, 3H, NCH₃), 2.27-2.39; (m, 4H, CH₂-3,4 (Pyr)). ¹⁹F NMR (Jun. 8 2018) (376.5 MHz, CDCl₃): −69.5
Electrochemical Impedance Spectroscopy (EIS)—The conductivities of the liquid and solid samples were measured following the procedure described by Makhlooghiazad et al, in J. Mater. Chem. A, 2017, 5, 5770, section 2.2.2, the content of which are hereby incorporated by reference.
Solid-state Nuclear Magnetic resonance spectroscopy (NMR)—Solid state NMR experiments were conducted on a commercial Bruker AVANCE III 500WB NMR spectrometer, using 2.5 mm zirconia rotors, following standard procedures as described in Mater. Adv., 2021, 2, page 1686, the content of which are hereby incorporated by reference.
Symmetrical Cell Cycling—Li symmetrical electrochemical coin cells were constructed to investigate the ability of the electrolytes to cycle Li metal with good efficiency and without breakdown using an electrolyte consisting of 10 or 50 mol % LiFSI in ZIPC1. They were cycled at 0.1 or 0.2 mA cm⁻² current density respectively at 50° C. for 1 hour for each polarization. A Biologic VMP3/Z potentiostat was used to cycle the cells galvanostatically and data was collected using EC-lab software version 11.27. The type of separator used for the cell cycling, transference number measurements and full cell cycling is specified in figure caption. The separators were dried under vacuum overnight and saturated by the liquid electrolyte (50 mol % LiFSI in ZIPC1). For the 10 mol % LiFSI in ZIPC1, the sample was melted at 90° C. then the separator was saturated by the melted electrolyte; after the separator was sufficiently wetted the temperature was decreased to 50° C. to solidify the electrolyte. These electrolytes were then sandwiched between two 8 mm diameter Li metal discs and assembled in a stainless steel cell case (Hohsen) using a 1 mm spacer and a 1.4 mm spring to provide uniform contact between electrodes and electrolyte inside the cell. Cell assembly was performed inside an argon-filled glove box. Cells were stored at 50° C. for 24 hours before being cycled.
Cyclic voltammetry—Cyclic voltammetry (CV) was performed to investigate the redox behaviour of Li in 10 mol % LiFSI in ZIPC1. CV was carried out with a two-electrode set up at a scan rate of 0.05 mV s⁻¹ at 50° C. using a Biologic VMP3/Z potentiostat driven by the EC-lab software. A glass fibre separator was saturated with the melted electrolyte, then it was sandwiched between a stainless steel working electrode and a Li metal disk (Sigma Aldrich) with 8 mm diameter as a reference/counter electrode and assembled in a stainless-steel coin cell. All the cell assembly processes were conducted under an argon atmosphere inside a glove box.
Transference number—Li symmetrical cells of 10 and 50 mol % LiFSI in ZIPC1 were prepared using the same process as for Li cycling tests and used to measure Li⁺ transference number at 50° C. using method described by Evans, Bruce, and Vincent. A small constant potential of 10 mV was applied to polarise cells and initial and steady state currents were determined. The impedance spectra were obtained before and after polarization. In order to obtain reproducible and reliable values several symmetric cells were made. The cells that showed either a very sharp increase in current or a short circuit were discarded, and the results reported are the average value from the others. A VMP3/Z Multi Potentiostat (Bio-Logic Science Instruments) and EC-Lab software version 11.27 was used for conducting all experiments and fitting the impedance data.
Full cell cycling—The cycling performance of the 10 mol % LiFSI in ZIPC1 was studied using 2032 coin-type cells with LiFePO₄ (LFP) cathode and Li metal disk (8 mm in diameter) as the anode at 50° C. with the lower and upper cut-off voltages of 2.8 and 3.8 V, respectively. The LFP cathode was fabricated by mixing 80 wt % of LFP powder, 10 wt % carbon black and 10 wt % polyvinylidene difluoride (PVDF) in N-methylpyrrolidone (NMP). The prepared slurry was coated uniformly on an aluminium current collector and dried overnight at room temperature. The cathode electrode was further dried in a vacuum oven at 110° C. for 16 h. The loading mass of the active material in the electrodes was ˜1.8 mg cm⁻². The electrolyte was prepared using the same process as for Li symmetrical cycling tests. The entire cell assembly process was performed inside an argon-filled glovebox. Cells were stored at 50° C. for 24 h prior to electrochemical tests, to ensure full absorption of the electrolyte into the electrodes. The galvanostatic charge-discharge studies were performed using a Biologic VMP-3 battery testing system at 50° C. inside an oven.

1. A zwitterionic plastic crystal (ZIPC) compound in the form of a non-polymeric molecule comprising:
at least one positively charged functional group carrying at least one positive charge, and
at least one negatively functional group carrying at least one negative charge, wherein
the positively charged functional groups and the negatively charged functional groups are covalently tethered together in the molecule, and the net charge of the zwitterionic compound is zero, and wherein the compound exhibits molecular disorder in the solid state, wherein the compound exhibits two or more of the following:
thermal phase behaviour which includes one or more solid-solid phase transitions before melting;
in the solid state one or more NMR linewidths of 20 KHz or less; and
a morphology including slip and glide planes on SEM analysis.

2. A zwitterionic plastic crystal (ZIPC) compound according to claim 1, wherein the compound exhibits three or more of the following:
thermal phase behaviour which includes one or more solid-solid phase transitions before melting;
in the solid state one or more NMR linewidths of 20 KHz or less;
a morphology including slip and glide planes on SEM analysis; and
long range, ordered crystal structure together with short-range disorder that originates from rotation or disorientation of the molecules within an ordered lattice; and
an entropy of fusion, ΔS_{f} of less than about 60 JK⁻¹ mol⁻¹.

3. A zwitterionic plastic crystal (ZIPC) compound according to claim 1, wherein at least one of the positive functional groups is derived from a cationic component comprising functional groups and/or substituents that facilitate delocalisation of the positive charge across the cationic moiety.

4. A zwitterionic plastic crystal (ZIPC) compound according to claim 1, wherein the unsubstituted or substituted heterocyclic ring cation is an imidazolium cation, pyridinium cation, pyrrolidinium cation, piperidinium cation, morpholinium cation, oxazolidinium cation, pyrazolium cation, triazolium cation, thiazolium cation, thiolanium cation, ammonium cation, guanidinium cation or thianium cation.

5. (canceled)

6. A zwitterionic plastic crystal (ZIPC) compound according to claim 1, wherein anionic component comprises a borate, a cyanoborate, a sulfonylimide, a fluorosulfonylimide (FSI), a hexafluorophoshate tetrafluoroborate, or a bis(trifluoromethanesulfonyll)imide (TFSA).

7. (canceled)

8. A zwitterionic plastic crystal (ZIPC) compound according to claim 1, derived from tethered cations and anions of a known OIPC selected from the group consisting of: [N_{1,1,1,1}][DCA], [N_{1,2,2,2}][BF₄], [P_{1,2,2,2}][TFS₁], [hexamethylguanidinium][TFSI], [hexamethylguanidinium][BF₄], [hexamethylguanidinium][FSI], [C₂mpyr][FSI], [C₂mpyr][BF₄], [P_{1,2,2,2}][FSI], [P_{1,2,2,i4}][PF₆], [P_{1,4,4,4}][FSI], [H₂im][Tf], [Hmim][Tf], [N_{2,2,3,3}][BBu₄], [N_{3,3,3,3}][BF₄], [C₂epyr][TFSI], [C₂epyr][FSI], [C₂epyr][PF₆], [C₂epyr][BF₄], [C₁moxa][FSI], [C₂moxa][FSI], [C₁moxa][TFSI] (oxa=oxazolidinium), [C₂mmor][FSI], [C₂mmor][TFSI], [C₂mmor][BF₄] (mor=morpholinium), [C₁₀₁mpyr][FSI], [C₂mpyr][TCM], [C₂mpyr][DFTFSI], [C₂mpyr][FTFSI], [C₁mpyr][(FH)₂F] and [C₂mpyr][(FH)₂F], [C₄mpyr][TFSI], [(NH₂)₃][Tf], [2-Me-im][Tf], and [TAZm][PFBS].

9. A zwitterionic plastic crystal (ZIPC) compound according to claim 1, in a protonated form.

10. A zwitterionic plastic crystal (ZIPC) compound according to claim 7, having one of the following protonated structures:

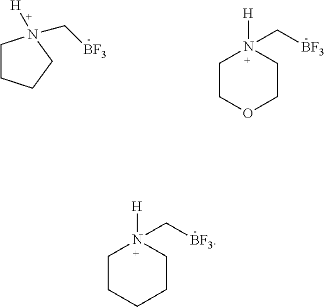

11. (canceled)

12. A zwitterionic plastic crystal (ZIPC) compound according to claim 1, exhibiting molecular disorder in the solid state, having one of the following general structures: 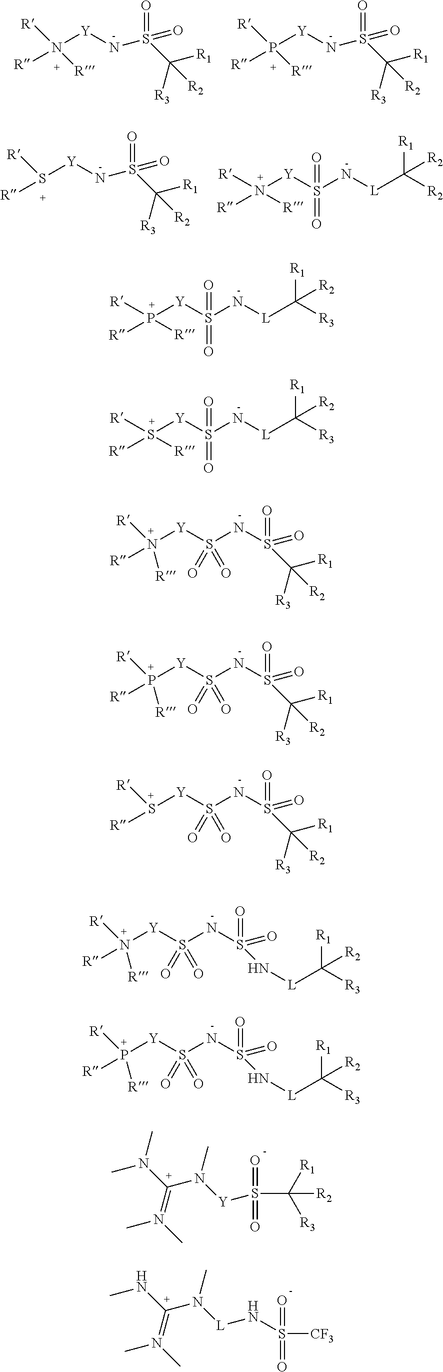 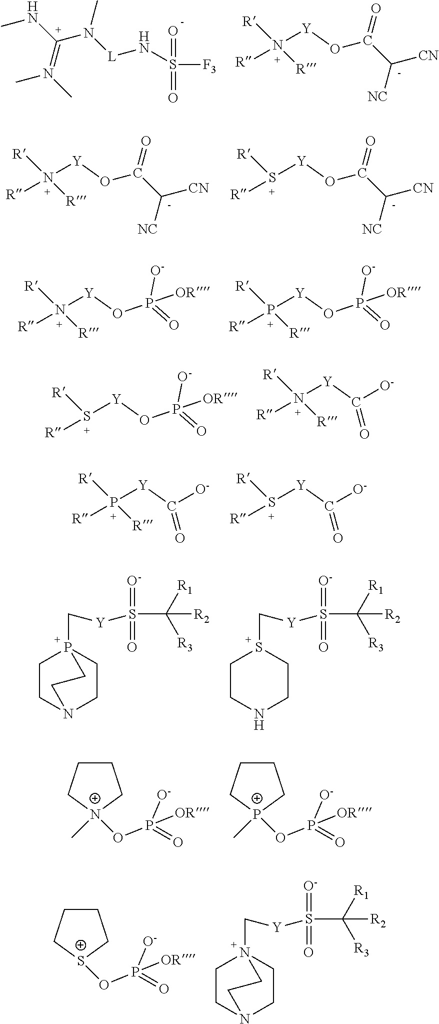
wherein:
one or more of R′, R″ and R″′ are independently selected from H, or an optionally substituted C₁₋₆alkyl, an optionally substituted fluoroC₁₋₆alkyl or a halo group, or one of R′ and R″, R″ and R″′ or R′ and R″′ form an optionally substituted 5- or 6-membered saturated or unsaturated heterocyclic ring,
each of R¹, R², and R³ are independently selected from H, an optionally substituted C₁₋₆alkyl, optionally substituted fluoroC₁₋₆alkyl, or a halo;
Y is N or an optionally substituted C₁₋₆ alkyl;
L is an optionally substituted C₁₋₆alkyl; and
wherein the optional substituents are selected from one or more of C₁₋₆ alkyl, halo, CN; OMe; or OEt.

13. A zwitterionic plastic crystal (ZIPC) compound according to claim 1, exhibiting molecular disorder in the solid state, having one of the following structures:

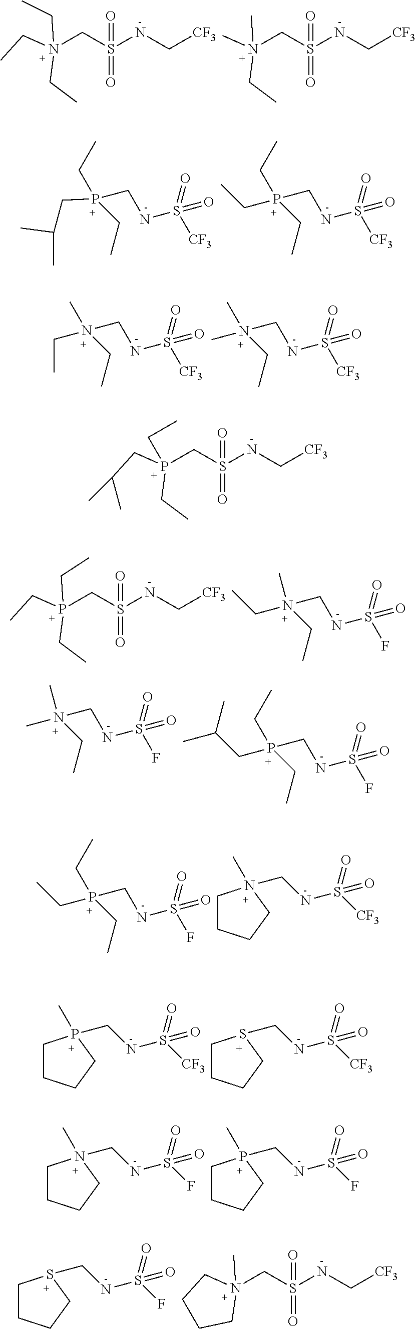

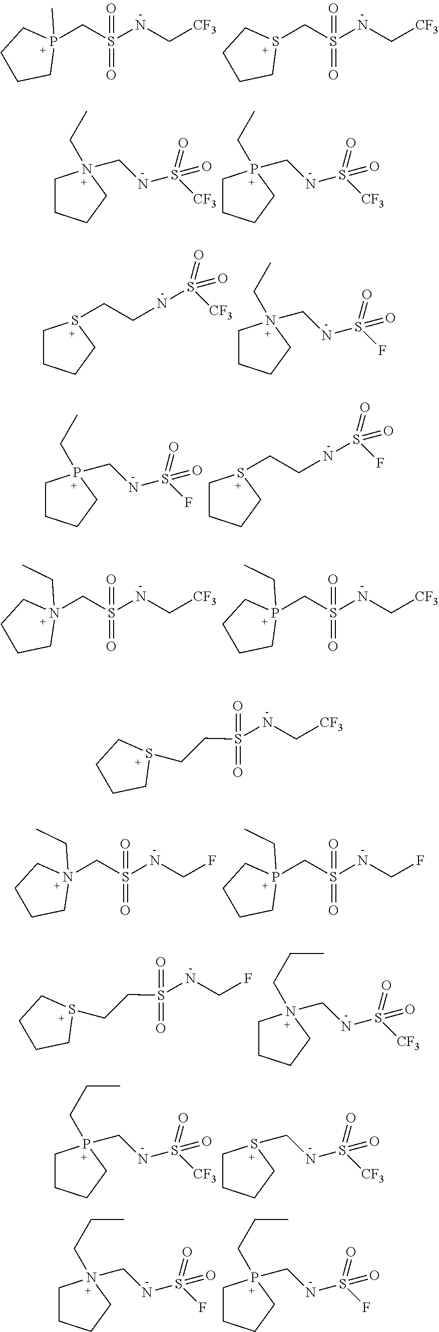

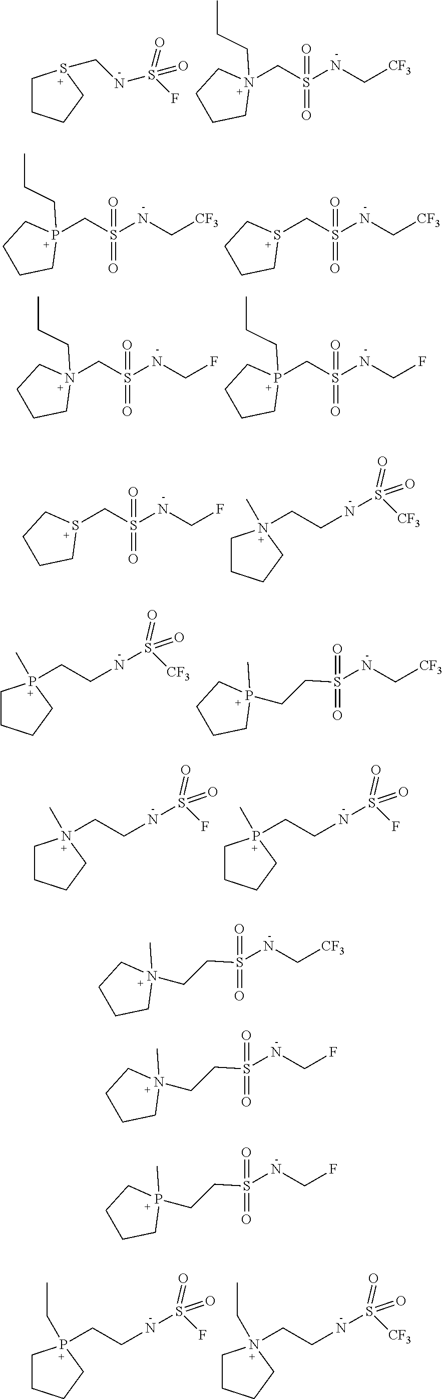

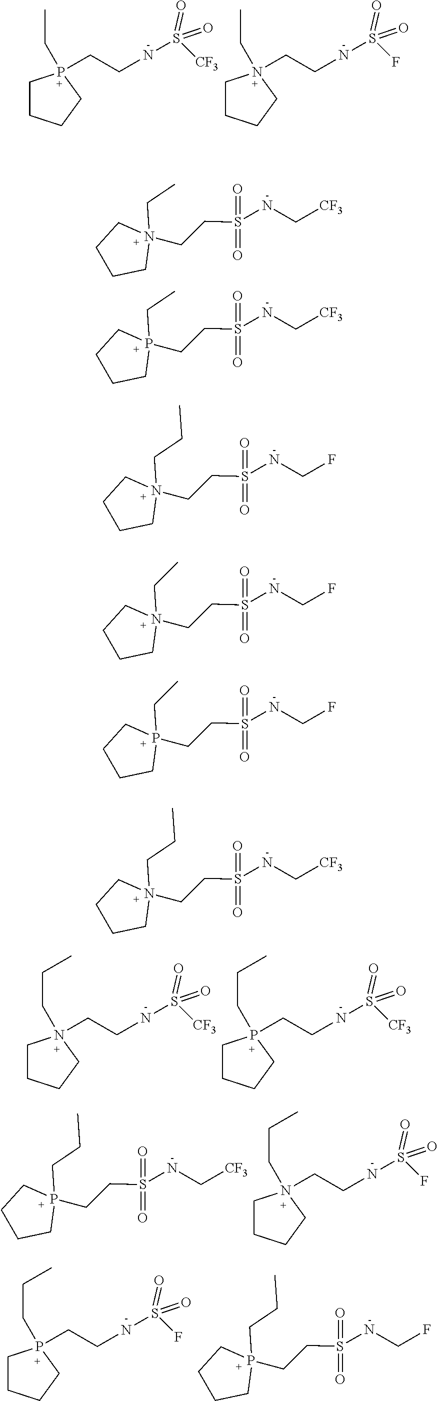

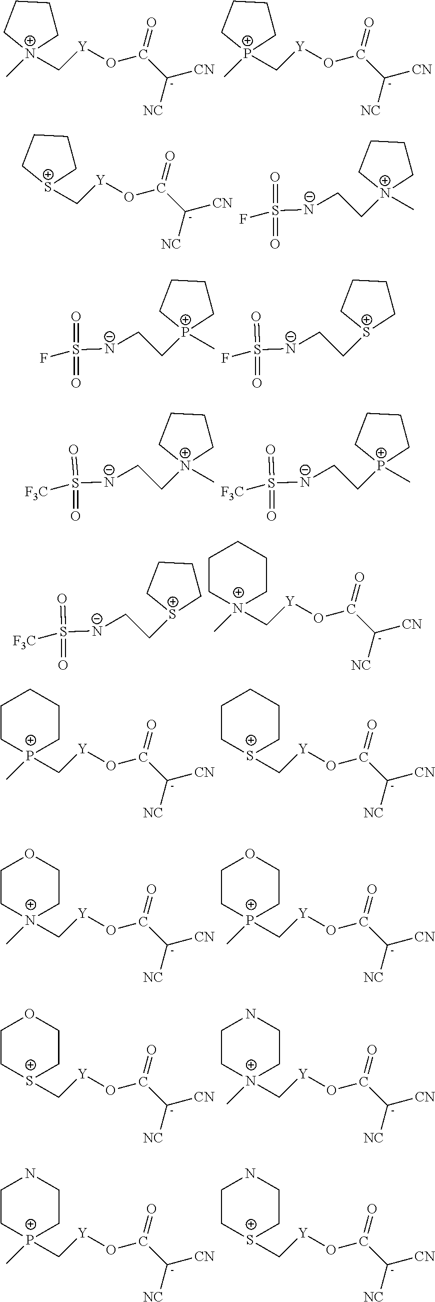

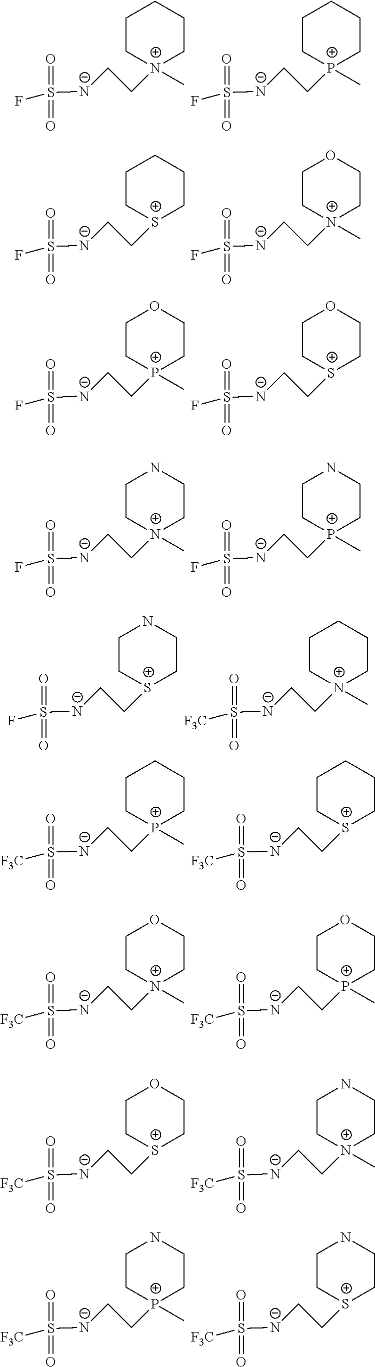

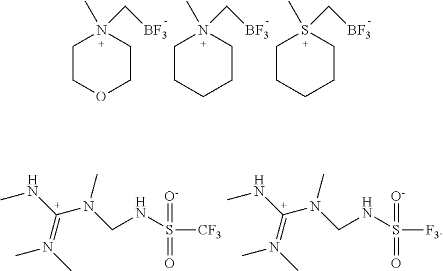

14. A zwitterionic plastic crystal (ZIPC) compound according to claim 1, exhibiting molecular disorder in the solid state, having one of the following structures:

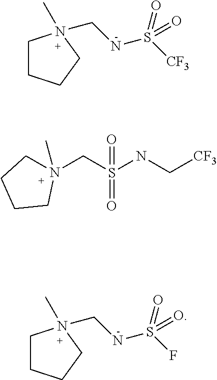

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. A zwitterionic plastic crystal (ZIPC) composition
in liquid form comprising a zwitterionic plastic crystal (ZIPC) compound according to claim 1, and an ionic salt, an acid, a base, a Li or Na functionalised polymer or combinations thereof;
or
in a solid-state form comprising a zwitterionic plastic crystal (ZIPC) compound according to claim 1, and an ionic salt, an acid, a base or combinations thereof.

29. (canceled)

30. A zwitterionic plastic crystal (ZIPC) composition according to claim 12, wherein the ZIPC is present in a concentration of at least 5 mol %.

31. A zwitterionic plastic crystal (ZIPC) composition according to claim 12, wherein the ZIPC is present in a concentration of 10 mol % or 90 mol %.

32. A zwitterionic plastic crystal (ZIPC) composition according to claim 12, wherein the ionic salt is one or more of an alkali metal salt, an alkaline earth metal salt, or a transition metal salt.

33. A zwitterionic plastic crystal (ZIPC) composition according to claim 12, wherein the ionic salt is one or more of LiBF₄, LiFSI, LiNTf₂, lithium bis(trifluoromethanesulfonyl)imide (Li[TFSI]), lithium (bis(fluorosulfonyl)imide (Li[FSI]), lithium triflate (Li[OTf]), lithium perchlorate (LiClO4), lithium dicyanamide (LiDCA), lithium cyanate (LiOCN), lithium bis[(pentafluoro-ethyl)sulfonyl]imide, lithium 2,2,2-trifluoromethylsulfonyl-N-cyanoamide (TFSAM), lithium 2,2,2-trifluoro-N-(trifluoromethylsulfonyl) acetamide (TSAC), lithium nonafluorobutanesulfonate (NF), lithium carborane, and lithium difluoro(oxolato)borate.

34. A zwitterionic plastic crystal (ZIPC) composition according to claim 12, wherein the acid is triflic acid.

35. A zwitterionic plastic crystal (ZIPC) composition according to claim 12, wherein the base is imidazole.

36. (canceled)

37. (canceled)

38. (canceled)

39. (canceled)

40. A solid-state electrolyte comprising a zwitterionic plastic crystal (ZIPC) compound according to claim 1 or a solid-state composition according to claim 12.

41. (canceled)

42. (canceled)

43. (canceled)

44. (canceled)

45. A zwitterionic plastic crystal (ZIPC) composition according to claim 15, wherein the ionic salt is a lithium salt or a sodium salt.
